(19) United States
(12) Patent Application Publication
(10) Pub. No.: US 2020/0399212 A1
(43) Pub. Date: Dec. 24, 2020

(54) SALT THAT INCLUDES AN ANION CONTAINING AN UNFLUORINATED DIALKYLAMIDE SULFONYL AND/OR SULFOXIMIDE GROUP AND CONTAINING A PERFLUOROALKYL SULFONYL GROUP

(71) Applicant: Robert Bosch GmbH, Stuttgart (DE)

(72) Inventors: Eduardo Sanchez Diez, Leioa (Vizcaya) (ES); Hany Basam Eitouni, Oakland, CA (US); Heng Zhang, Vitoria-Gasteiz Araba (ES); Ismael Gracia, Zaragoza (ES); Maria Martinez Ibanez, Vitoria-Gasteiz (ES); Mario Joost, Leonberg (DE); Michel Armand, Paris (FR); Uxue Oteo, Bilbao (ES)

(21) Appl. No.: 16/902,221

(22) Filed: Jun. 15, 2020

(30) Foreign Application Priority Data
Jun. 19, 2019 (DE) 102019208914.3

(51) Int. Cl. C07C 311/09; H01M 2/16; H01M 10/0525; H01M 10/0565

(52) U.S. Cl. CPC C07C 311/09; H01M 10/0565; H01M 10/0525; H01M 2/1653

(57) ABSTRACT

A salt, in particular a lithium salt, that is usable in alkali metal cells and/or batteries, and/or electrolytes and/or additives for same is described. To achieve good ion conductivity as well as temperature and high voltage stability and to prevent corrosion of aluminum, for example a current collector, the salt includes at least one alkali metal ion and/or at least one ammonium compound and at least one anion that contains at least one unfluorinated (N,N-dialkylamide)sulfonyl group and/or at least one unfluorinated (N,N-dialkylamide)sulfoximide group, and at least one (perfluoroalkyl)sulfonyl group. An electrolyte, an additive, a cell, a battery, and a preparation method, are also described.

### CROSS REFERENCE

The present application claims the benefit under 35 U.S.C. § 119 of German Patent Application No. DE 102019208914.3 filed on Jun. 19, 2019, which is expressly incorporated herein by reference in its entirety.

### FIELD

The present invention relates to a salt, in particular a lithium salt that is usable in alkali metal cells and/or batteries, and/or electrolytes and/or additives for same, and an electrolyte, an additive, a cell, a battery, and a preparation method.

### BACKGROUND INFORMATION

Polymer electrolyte lithium-ion cells and/or batteries may have high energy densities, for example >400 Wh/kg, and a high level of safety, in particular due to avoidance of liquid electrolytes.

It is of particular interest to use high-voltage cathode active materials in polymer electrolyte lithium-ion cells and/or batteries. However, most conventional lithium conducting salts have only limited electrochemical stability in the presence of high-voltage cathode active materials.

Problems with corrosion of an aluminum current collector are described in the scientific publication Journal of Power Sources 68 (997) 320. In addition, problems may occur with formation of a stable protective layer and with stability at high temperatures.

To solve these problems, it has been provided to use additional solvents, such as nitrogen-containing polar solvents, or methyl difluoroacetate (Electrochemistry Communications 7 (2005) 1000; Electrochemical and Solid-State Letters, 8 (2005) A459), to modify conventional lithium conducting salts such as lithium bis(trifluoromethanesulfonyl)imide (LiTFSI; IUPAC: lithium bis[(trifluoromethyl)sulfonyl]azanide) or lithium hexafluorophosphate (LiPF₆) (Electrochemistry Communications 13 (2011) 265; Electrochimica Acta 114 (2013) 95), or to develop novel salts (Electrochemical and Solid-State Letters, 11 (2008) C19; Electrochemical and Solid-State Letters, 12 (2009) A155; Electrochimica Acta 55 (2010) 1450; Journal of Power Sources 195 (2010) 5344)), or to add ionic liquids as plasticizers (Electrochemistry Communications 5 (2003) 1016).

U.S Patent Application Publication Nos. US 2014/154591 A1, US 2014/272601 A1, and US 2008/138715 A1 describe lithium conducting salts.

### SUMMARY

The present invention relates to a salt that includes at least one alkali metal ion, in particular at least one alkali metal cation, and/or at least one ammonium compound, in particular at least one ammonium compound cation, and at least one anion.

In accordance with the present invention, the at least one anion contains in particular
at least one unfluorinated, for example unhalogenated, (N,N-dialkylamide)sulfonyl group, in particular S—(N,N-dialkylamide)sulfonyl group, and/or
at least one unfluorinated, for example unhalogenated, (N,N-dialkylamide)sulfoximide group, in particular S—(N,N-dialkylamide)sulfoximide group, and
at least one (perfluoroalkyl)sulfonyl group, in particular S-(perfluoroalkyl)sulfonyl group.

### BRIEF DESCRIPTION OF THE DRAWING

FIG. 1 shows a schematic graph for depicting the lithium ion conductivity of mixtures 11, 11* of lithium [(trifluoromethyl)sulfonyl][(methylethylamide)sulfonyl]azanide, prepared via two different synthesis routes, and polyethylene oxide with a concentration of 1 lithium ion per 20 ethylene oxide units as a function of the temperature, compared to a corresponding mixture of lithium bis[(trifluoromethyl)sulfonyl]azanide (LiTFSI) in polyethylene oxide 10 under the same conditions.

### DETAILED DESCRIPTION OF EXAMPLE EMBODIMENTS

The present invention relates to a salt that includes at least one alkali metal ion, in particular at least one alkali metal cation, and/or at least one ammonium compound, in particular at least one ammonium compound cation, and at least one anion.

In accordance with the present invention, the at least one anion contains in particular
at least one unfluorinated, for example unhalogenated, (N,N-dialkylamide)sulfonyl group, in particular S—(N,N-dialkylamide)sulfonyl group, and/or
at least one unfluorinated, for example unhalogenated, (N,N-dialkylamide)sulfoximide group, in particular S—(N,N-dialkylamide)sulfoximide group, and
at least one (perfluoroalkyl)sulfonyl group, in particular S-(perfluoroalkyl)sulfonyl group.

An ammonium compound, in particular an ammonium compound cation, may be understood in particular to mean a compound containing at least one positively charged nitrogen atom with four bonding partners bound thereto. The four bonding partners may in particular independently be organic functional groups or hydrogen.

An unfluorinated, for example unhalogenated, (N,N-dialkylamide)sulfonyl group, in particular S—(N,N-dialkylamide)sulfonyl group, may be understood in particular to mean a functional group of the general chemical formula:

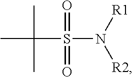

where R1 and R2 each independently stand for an unfluorinated, for example unhalogenated, alkyl group. [symbol] stands in particular for a bonding site or bond via which the unfluorinated, for example unhalogenated, (N,N-dialkylamide)sulfonyl group, in particular S—(N,N-dialkylamide)sulfonyl group, may be bound to a substituent.

A sulfoximide group (see IUPAC Rule C-633.1), which may also be referred to as a sulfoximine group, may be understood in particular to mean a functional group that is derivable from the >S(═O)═N— structural unit.

An unfluorinated, for example unhalogenated, (N,N-dialkylamide)sulfoximide group, in particular S—(N,N-dialkylamide)sulfoximide group, may be understood in particular to mean a functional group of the general chemical formula:

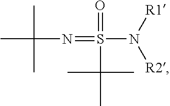

where R1′ and R2′ each independently stand for an unfluorinated, for example unhalogenated, alkyl group. [symbol] in each case in particular stands for a bonding site or bond via which the unfluorinated, for example unhalogenated, (N,N-dialkylamide)sulfoximide group, in particular S—(N,N-dialkylamide)sulfoximide group, may in each case be bound to a substituent. The unfluorinated, for example unhalogenated, (N,N-dialkylamide)sulfoximide group, in particular S—(N,N-dialkylamide)sulfoximide group, may thus be in particular divalent and/or bridging.

A (perfluoroalkyl)sulfonyl group, in particular S-(perfluoroalkyl)sulfonyl group, may be understood in particular to mean a functional group of the general chemical formula:

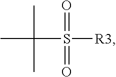

where R3 stands for a perfluorinated alkyl group. [symbol] in particular stands for a bonding site or bond via which the (perfluoroalkyl)sulfonyl group, in particular S-(perfluoroalkyl)sulfonyl group, may be bound to a substituent.

The at least one anion may be characterized in particular by enhanced delocalization of the negative charge and thus, by a reduced charge density. Good dissociation and thus good ion conductivity may thus be advantageously achieved.

Due to the reduced charge density, the salt may also have a reduced melting point of less than 190° C., for example. As a result of the low melting point, the salt, in particular provided that it is liquid in the operating temperature window of the cell, may, for example in the form of ionic liquids, also be used as a plasticizer for polymers, for example for a polymer electrolyte, and may thus also increase the ion conductivity.

In addition, the salt may advantageously be stable with respect to in particular metallic lithium (Li⁺/Li) at elevated temperatures, for example 200° C., and/or at high cell voltages, for example 4.5 V.

Furthermore, the salt may also advantageously show at least greatly reduced or even no corrosion, in particular aluminum corrosion, of a current collector, for example, which may indicate reduced acidity.

Overall, a salt may thus be advantageously provided that has good ion conductivity, is stable at high temperature and high voltage, and results in hardly or in particular no corrosion of aluminum.

The salt may therefore be particularly advantageously used for this purpose in alkali metal cells and/or batteries, for example in alkali metal-ion cells and/or batteries, for example in lithium cells and/or batteries and/or in sodium cells and/or batteries, in particular in lithium cells and/or batteries, for example in lithium-ion cells and/or batteries and/or in sodium-ion cells and/or batteries, in particular in lithium-ion cells and/or batteries, for example in the form of solid electrolyte cells and/or batteries, for example in the form of polymer electrolyte cells and/or batteries, for example in the form of solid-state cells and/or batteries, for example in so-called all-solid-state cells and/or batteries, in particular having a high energy density and/or including a high-voltage cathode active material and/or having an elevated operating temperature, and/or may be used in electrolyte and/or additives.

Within the scope of one specific embodiment, the at least one anion has the general chemical formula:

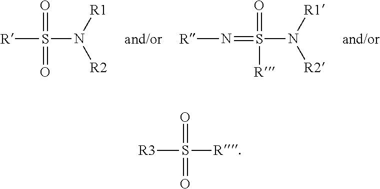

R1 and R2 may in particular each independently stand for an unfluorinated, for example unhalogenated, alkyl group.

R1′ and R2′ may in particular each independently stand for an unfluorinated, for example unhalogenated, alkyl group.

R3 may in particular stand for a perfluorinated alkyl group.

R′ may in particular stand for a substituent that includes at least one (perfluoroalkyl)sulfonyl group, in particular S-(perfluoroalkyl)sulfonyl group.

R″ may in particular stand for a substituent that includes at least one (perfluoroalkyl)sulfonyl group, in particular S-(perfluoroalkyl)sulfonyl group, where R′″ may stand for a substituent that in particular includes at least one, in particular unfluorinated, for example unhalogenated, (N,N-dialkylamide)sulfoximide group, in particular S—(N,N-dialkylamide)sulfoximide group, and/or at least one, in particular unfluorinated, for example unhalogenated, (N,N-dialkylamide)sulfonyl group, in particular S—(N,N-dialkylamide)sulfonyl group, for example at least one, in particular unfluorinated, for example unhalogenated, (N,N-dialkylamide)sulfoximide group, in particular S—(N,N-dialkylamide)sulfoximide group, and at least one, in particular unfluorinated, for example unhalogenated, (N,N-dialkylamide)sulfonyl group, in particular S—(N,N-dialkylamide)sulfonyl group, and/or at least one (perfluoroalkyl)sulfonyl group, in particular S-(perfluoroalkyl)sulfonyl group. For example, R″ may stand for a substituent that includes at least one (perfluoroalkyl)sulfonyl group, in particular S-(perfluoroalkyl)sulfonyl group, where R′″ may stand for a substituent that includes at least one, in particular unfluorinated, for example unhalogenated, (N,N-dialkylamide)sulfoximide group, in particular S—(N,N-dialkylamide)sulfoximide group, and at least one (perfluoroalkyl)sulfonyl group, in particular S-(perfluoroalkyl)sulfonyl group.

Alternatively, R″ may, for example, stand for a substituent that in particular includes at least one, in particular unfluorinated, for example unhalogenated, (N,N-dialkylamide)sulfoximide group, in particular S—(N,N-dialkylamide)sulfoximide group, and/or at least one, in particular unfluorinated, for example unhalogenated, (N,N-dialkylamide)sulfonyl group, in particular S—(N,N-dialkylamide)sulfonyl group, for example at least one, in particular unfluorinated, for example unhalogenated, (N,N-dialkylamide)sulfoximide group, in particular S—(N,N-dialkylamide)sulfoximide group, and at least one, in particular unfluorinated, for example unhalogenated, (N,N-dialkylamide)sulfonyl group, in particular S—(N,N-dialkylamide)sulfonyl group, and/or at least one (perfluoroalkyl)sulfonyl group, in particular S-(perfluoroalkyl)sulfonyl group, where R′″ may stand for a substituent that includes at least one (perfluoroalkyl)sulfonyl group, in particular S-(perfluoroalkyl)sulfonyl group.

R″″ may in particular stand for a substituent that includes at least one, in particular unfluorinated, for example unhalogenated, (N,N-dialkylamide)sulfonyl group, in particular S—(N,N-dialkylamide)sulfonyl group, and/or at least one, in particular unfluorinated, for example unhalogenated, (N,N-dialkylamide)sulfoximide group, in particular S—(N,N-dialkylamide)sulfoximide group.

R′ and/or R″ and/or R′″ and/or R″″, in particular R′ and/or R″ or R′″ and/or R″″, may, for example, in particular independently of one another in each case, stand for a substituent that additionally includes at least one, for example bridging, nitrogen atom, for example in the form of an in particular negatively charged azanide group, or in the form of a tertiary amine group, for example in the form of an in particular negatively charged azanide group. The at least one (perfluoroalkyl)sulfonyl group, in particular S-(perfluoroalkyl)sulfonyl group, or the at least one, in particular unfluorinated, for example unhalogenated, (N,N-dialkylamide)sulfoximide group, in particular S—(N,N-dialkylamide)sulfoximide group, and/or the at least one, in particular unfluorinated, for example unhalogenated, (N,N-dialkylamide)sulfonyl group, in particular S—(N,N-dialkylamide)sulfonyl group, may be bound, for example via the additional at least one, for example bridging, nitrogen atom, for example in the form of an in particular negatively charged azanide group or in the form of a tertiary amine group, for example in the form of an in particular negatively charged azanide group.

An in particular negatively charged azanide group may be understood in particular to mean a functional group of the general chemical formula:

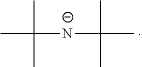

[symbol] in each case in particular stands for a bonding site or bond via which the in particular negatively charged azanide group in each case may be bound to a substituent. The in particular negatively charged azanide group may thus in particular be divalent and/or bridging.

In particular, R′ and/or R″ and/or R′″ and/or R″″, for example R′ and/or R″ or R′″ and/or R″″, in each case may in particular independently stand for a substituent having at least one negative charge. R″ and R′″ may in particular stand for substituents, of which at least one, optionally also both, has/have at least one negative charge.

Within the scope of another specific embodiment, R′ and/or R″ and/or R′″ and/or R″″, in particular R′ and/or R″ or R′″ and/or R″″, includes(s) at least one negative charge in the form of an in particular negatively charged azanide group, for example in the form of a bridging, in particular negatively charged, azanide group.

Within the scope of another specific embodiment, the salt includes or is an alkali metal salt. The alkali metal salt, in contrast to conventional ionic liquids, may thus provide ions, for example lithium ions, for the ion conductivity, thus increasing the ion conductivity due to the plasticizer effect and also in particular by providing ions, for example lithium ions.

Within the scope of one embodiment of this specific embodiment, the salt includes or is a lithium salt that contains at least one lithium ion. Thus, the salt may be particularly advantageously used as a lithium conducting salt and/or an additive in lithium cells and/or batteries. The salt preferably includes or is a lithium salt that contains at least one lithium ion.

Within the scope of another alternative or additional embodiment of this specific embodiment, the salt includes or is a potassium salt that contains at least one potassium ion. Potassium salts may be easily synthesized. The potassium salt may advantageously be used as such as an additive, for example, also in a lithium cell and/or battery, for example. In addition, the potassium salt may be easily converted into a lithium salt by ion exchange. The lithium salt may then in turn advantageously be used as a lithium conducting salt and/or as an additive, for example in a lithium cell and/or battery.

Within the scope of another alternative or additional embodiment of this specific embodiment, the salt includes or is a sodium salt that contains at least one sodium ion. The salt may thus be particularly advantageously used as a sodium conducting salt and/or additive in sodium cells and/or batteries.

Within the scope of another alternative or additional specific embodiment, the salt includes or is an ammonium compound salt that contains at least one ammonium compound, in particular as a cation.

An ammonium compound salt may be understood in particular to mean a salt that includes at least one ammonium compound as cation.

Ammonium compound salts may advantageously have a low melting point and/or may be ionic liquids and used as plasticizers for polymers, for example for a polymer electrolyte, for example of a separator and/or a cathode, and/or may increase the ion conductivity.

Within the scope of one embodiment of this specific embodiment, the salt includes or is a salt of an organic ammonium compound that includes at least one organic ammonium compound, in particular as cation. For example, the salt may be a dialkylammonium salt.

Within the scope of another, in particular alternative or additional, embodiment of this specific embodiment, the salt includes or is an ammonium salt that contains at least one ammonium ion (NH₄⁺).

Within the scope of another specific embodiment, R1 and R2 each independently stand for an unfluorinated, in particular unhalogenated, methyl group or an unfluorinated, in particular unhalogenated, ethyl group or an unfluorinated, in particular unhalogenated, propyl group, for example an unfluorinated, in particular unhalogenated, n-propyl group or an unfluorinated, in particular unhalogenated, isopropyl group, or an unfluorinated, in particular unhalogenated, butyl group, for example an unfluorinated, in particular unhalogenated, n-butyl group or an unfluorinated, in particular unhalogenated, isobutyl group or an unfluorinated, in particular unhalogenated, t-butyl group. Within the scope of one embodiment of this specific embodiment, R1 and R2 each independently stand for an unfluorinated, in particular unhalogenated, methyl group or for an unfluorinated, in particular unhalogenated, ethyl group.

Within the scope of another alternative or additional specific embodiment, R1′ and R2′ each independently stand for an unfluorinated, in particular unhalogenated, methyl group or for an unfluorinated, in particular unhalogenated, ethyl group or for an unfluorinated, in particular unhalogenated, propyl group, for example for an unfluorinated, in particular unhalogenated, n-propyl group or for an unfluorinated, in particular unhalogenated, isopropyl group, or for an unfluorinated, in particular unhalogenated, butyl group, for example for an unfluorinated, in particular unhalogenated, n-butyl group or for an unfluorinated, in particular unhalogenated, isobutyl group or for an unfluorinated, in particular unhalogenated, t-butyl group.

Within the scope of one embodiment of this specific embodiment, R1′ and R2′ each independently stand for an unfluorinated, in particular unhalogenated, methyl group or for an unfluorinated, in particular unhalogenated, ethyl group.

Within the scope of another specific embodiment, R1 and R2 stand for different unfluorinated, in particular unhalogenated, alkyl groups. The crystallization tendency and/or the melting point may advantageously be reduced due to an asymmetrical configuration. As described above, this may in turn have an advantageous effect on the ion conductivity and/or on plasticizer properties and/or achieving a liquid state of matter. For example, R1 may stand for an unfluorinated, in particular unhalogenated, methyl group and R2 may stand for an unfluorinated, in particular unhalogenated, ethyl group.

Within the scope of another alternative or additional specific embodiment, R1 and R2 stand for the same unfluorinated, in particular unhalogenated, alkyl groups. The synthesis may advantageously be further simplified in this way. For example, R1 and R2 may each stand for an unfluorinated, in particular unhalogenated, methyl group, or R1 and R2 may each stand for an unfluorinated, in particular unhalogenated, ethyl group.

Within the scope of another alternative or additional specific embodiment, R1′ and R2′ stand for different unfluorinated, in particular unhalogenated, alkyl groups. The crystallization tendency and/or the melting point may advantageously be reduced due to an asymmetrical configuration. As described above, this may in turn have an advantageous effect on the ion conductivity and/or on plasticizer properties and/or achieving a liquid state of matter. For example, R1′ may stand for an unfluorinated, in particular unhalogenated, methyl group and R2′ may stand for an unfluorinated, in particular unhalogenated, ethyl group.

Within the scope of another alternative or additional specific embodiment, R1′ and R2′ stand for the same unfluorinated, in particular unhalogenated, alkyl groups. The synthesis may advantageously be further simplified in this way. For example, R1′ and R2′ may each stand for an unfluorinated, in particular unhalogenated, methyl group, or R1′ and R2′ may each stand for an unfluorinated, in particular unhalogenated, ethyl group.

Within the scope of another specific embodiment, R3 stands for a perfluorinated methyl group, in particular a trifluoromethyl group (—CF₃), or for a perfluorinated ethyl group, in particular for a pentafluoroethyl group (—C₂F₅) or for a perfluorinated propyl group, in particular for a perfluorinated n-propyl group or for a perfluorinated isopropyl group, for example for an n-heptafluoropropyl or isoheptafluoropropyl group (-n-C₃F₇ or -iso-C₃F₇), or for a perfluorinated butyl group, in particular for a perfluorinated n-butyl group or for a perfluorinated isobutyl group or for a perfluorinated t-butyl group, for example for an n-heptafluoropropyl or isoheptafluoropropyl or t-heptafluoropropyl group (-n-C₄F₉ or -iso-C₄F₉ or -t-C₄F₉).

In one particular embodiment of this specific embodiment, R3 stands for a perfluorinated methyl group, in particular a trifluoromethyl group (—CF₃).

Within the scope of another specific embodiment, the salt or the at least one anion includes
at least two unfluorinated, for example unhalogenated, (N,N-dialkylamide)sulfonyl groups, in particular S—(N,N-dialkylamide)sulfonyl groups, and/or
at least two unfluorinated, for example unhalogenated, (N,N-dialkylamide)sulfoximide groups, in particular S—(N,N-dialkylamide)sulfoximide groups, and/or
at least two (perfluoroalkyl)sulfonyl groups, for example at least two S-(perfluoroalkyl)sulfonyl groups.

The charge may thus be even better delocalized and/or the melting point may be further lowered, thus further improving the ion conductivity and/or the plasticizer properties. For example, the salt or the at least one anion may include at least two unfluorinated, for example unhalogenated, (N,N-dialkylamide)sulfoximide groups, in particular S—(N,N-dialkylamide)sulfoximide groups, for example at least two unfluorinated, for example unhalogenated, (N,N-dialkylamide)sulfoximide groups, in particular S—(N,N-dialkylamide)sulfoximide groups, and at least two (perfluoroalkyl)sulfonyl groups, for example at least two S-(perfluoroalkyl)sulfonyl groups.

Within the scope of another specific embodiment, the at least one anion has the general chemical formula:

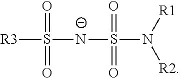

For example, the salt may include or be an alkali metal [(perfluoroalkyl)sulfonyl][(dialkylamide)sulfonyl]azanide and/or a dialkylammonium [(perfluoroalkyl)sulfonyl][(dialkylamide)sulfonyl]azanide.

Within the scope of one embodiment of this specific embodiment, the at least one anion has the general chemical formula:

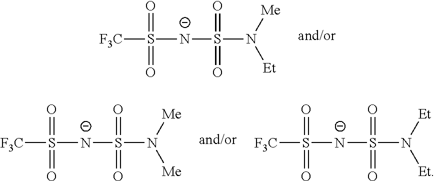 [SMILES: CCN(C)S(=O)(=O)[N-]S(=O)(=O)C(F)(F)F ; CN(C)S(=O)(=O)[N-]S(=O)(=O)C(F)(F)F ; CCN(CC)S(=O)(=O)[N-]S(=O)(=O)C(F)(F)F]

Me may in particular stand for an unfluorinated, for example unhalogenated, methyl group, in particular —CH₃.

Et may in particular stand for an unfluorinated, for example unhalogenated, ethyl group, in particular —C₂H₅.

For example, the salt may include or be an alkali metal [(trifluoromethyl)sulfonyl][(methylethylamide)sulfonyl]azanide and/or an alkali metal [(trifluoromethyl)sulfonyl][(dimethylamide)sulfonyl]azanide and/or an alkali metal [(trifluoromethyl)sulfonyl][(diethylamide)sulfonyl]azanide and/or a methylethylammonium [(trifluoromethyl)sulfonyl][(methylethylamide)sulfonyl]azanide and/or a dimethylammonium [(trifluoromethyl)sulfonyl][(dimethylamide)sulfonyl]azanide and/or a diethylammonium [(trifluoromethyl)sulfonyl][(diethylamide)sulfonyl]azanide.

Within the scope of one particular embodiment of this specific embodiment, the salt includes or has the general chemical formula:

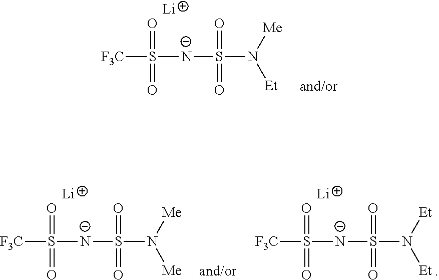 [SMILES: [Li+] ; CCN(C)S(=O)(=O)[N-]S(=O)(=O)C(F)(F)F ; [Li+] ; [Li+] ; CN(C)S(=O)(=O)[N-]S(=O)(=O)C(F)(F)F ; CCN(CC)S(=O)(=O)[N-]S(=O)(=O)C(F)(F)F]

The salt may be referred to in particular as lithium [(trifluoromethyl)sulfonyl][(methylethylamide)sulfonyl]azanide or as lithium [(trifluoromethyl)sulfonyl][(dimethylamide)sulfonyl]azanide or as lithium [(trifluoromethyl)sulfonyl][(diethylamide)sulfonyl]azanide.

Within the scope of another alternative or additional specific embodiment, the at least one anion has the general chemical formula:

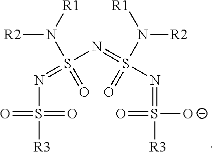

In particular due to the conjugated double bond system, a particularly high charge delocalization or a particularly low charge density may thus advantageously be achieved and/or the melting point may be further lowered, thus further improving the ion conductivity and/or the plasticizer properties.

Within the scope of one embodiment of this specific embodiment, the at least one anion has the general chemical formula:

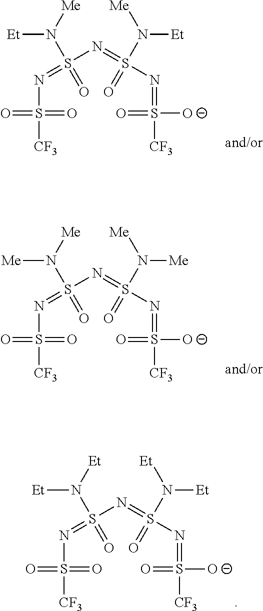 [SMILES: CCN(C)S(=O)(=NS(=O)(=O)C(F)(F)F)N=S(=O)(N=S(=O)([O-])C(F)(F)F)N(C)CC ; CN(C)S(=O)(=NS(=O)(=O)C(F)(F)F)N=S(=O)(N=S(=O)([O-])C(F)(F)F)N(C)C ; CCN(CC)S(=O)(=NS(=O)(=O)C(F)(F)F)N=S(=O)(N=S(=O)([O-])C(F)(F)F)N(CC)CC]

Me may in particular stand for an unfluorinated, for example unhalogenated, methyl group, in particular —CH₃.

Et may in particular stand for an unfluorinated, for example unhalogenated, ethyl group, in particular —C₂H₅.

Within the scope of one particular embodiment of this specific embodiment, the salt includes or has the general chemical formula:

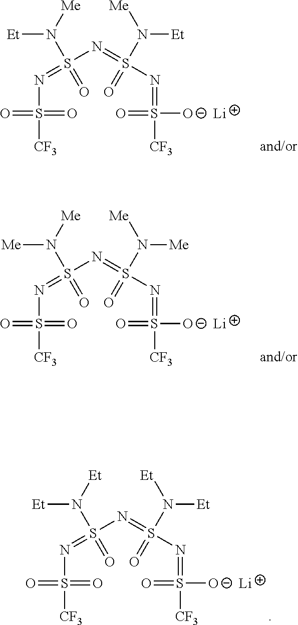 [SMILES: CCN(C)S(=O)(=NS(=O)(=O)C(F)(F)F)N=S(=O)(N=S(=O)([O-])C(F)(F)F)N(C)CC ; [Li+] ; CN(C)S(=O)(=NS(=O)(=O)C(F)(F)F)N=S(=O)(N=S(=O)([O-])C(F)(F)F)N(C)C ; [Li+] ; CCN(CC)S(=O)(=NS(=O)(=O)C(F)(F)F)N=S(=O)(N=S(=O)([O-])C(F)(F)F)N(CC)CC ; [Li+]]

The salts may be verified, for example, with the aid of nuclear magnetic resonance (NMR) spectroscopy and/or other analytical methods.

With regard to further technical features and advantages of the salt according to the present invention, explicit reference is hereby made to the explanations in conjunction with the electrolyte according to the present invention, the additive according to the present invention, the cell according to the present invention, the battery according to the present invention, and the preparation method according to the present invention, and to the FIGURE and the description of the FIGURE.

A further subject matter of the present invention relates to an electrolyte that includes at least one salt according to the present invention.

In particular, the electrolyte may be designed for an alkali metal cell and/or battery, in particular for an alkali metal-ion cell and/or battery, for example for a lithium cell and/or battery and/or for a sodium cell and/or battery, in particular for a lithium cell and/or battery, for example for a lithium-ion cell and/or battery and/or for a sodium-ion cell and/or battery, in particular for a lithium-ion cell and/or lithium-ion battery, for example in the form of a solid electrolyte cell and/or battery, for example in the form of a polymer electrolyte cell and/or battery, for example in the form of a solid-state cell and/or battery, such as in the form of a so-called all-solid-state cell and/or battery, for example having a high energy density and/or including a high-voltage cathode active material and/or having an elevated operating temperature.

For example, the electrolyte may include at least one lithium salt according to the present invention and/or at least one ammonium compound salt according to the present invention and/or at least one potassium salt according to the present invention and/or at least one sodium salt according to the present invention. For example, the electrolyte may include at least one lithium salt according to the present invention and/or at least one ammonium compound salt according to the present invention. In particular, the electrolyte may include at least one lithium salt according to the present invention.

The at least one salt according to the present invention may be used as a conducting salt and/or as an electrolyte additive.

Within the scope of one specific embodiment, the electrolyte includes at least one further salt. For example, the electrolyte may include at least one further lithium salt and/or at least one, optionally further, lithium conducting salt, for example lithium bis(trifluoromethanesulfonyl)imide (LiTFSI; IUPAC: lithium bis[(trifluoromethyl)sulfonyl]azanide) and/or lithium-4,5-dicyano-2-(trifluoromethyl)imidazole (LiTDI) and/or lithium tetrafluoroborate (LiBF₄) and/or lithium hexafluorophosphate (LiPF₆) and/or lithium bis(fluorosulfonyl)imide (Li[N(SO₂F)₂], LiFSI) and/or lithium bis(oxalato)borate (LiB(C₂O₄)₂, LiBOB) and/or lithium difluoro(oxalato)borate (Li[BF₂(C₂O₄)], LiDFOB).

In particular, the at least one salt according to the present invention may be used in combination with at least one, optionally further, lithium conducting salt and/or as a component of a salt mixture, for example a lithium conducting salt mixture, and/or as an electrolyte additive.

In principle, the electrolyte may be a liquid electrolyte or a gel electrolyte or a solid electrolyte, in particular a polymer electrolyte.

Within the scope of another specific embodiment, the electrolyte is a polymer electrolyte. For example, the electrolyte may be a polymer electrolyte that includes at least one polyalkylene oxide, for example polyethylene oxide (PEO). Polymer electrolytes may be used particularly advantageously in lithium cells.

With regard to further technical features and advantages of the electrolyte according to the present invention, explicit reference is hereby made to the explanations in conjunction with the salt according to the present invention, the additive according to the present invention, the cell according to the present invention, the battery according to the present invention, and the preparation method according to the present invention, and to the FIGURE and the description of the FIGURE.

Moreover, the present invention relates to an additive for an alkali metal cell and/or battery, in particular for an alkali metal-ion cell and/or battery, for example for a lithium cell and/or battery and/or for a sodium cell and/or battery, in particular for a lithium cell and/or battery, for example for a lithium-ion cell and/or battery and/or for a sodium-ion cell and/or battery, in particular for a lithium-ion cell and/or battery, that includes or is at least one salt according to the present invention, for example at least one lithium salt according to the present invention and/or at least one ammonium compound salt according to the present invention and/or at least one potassium salt according to the present invention and/or at least one sodium salt according to the present invention, for example at least one lithium salt according to the present invention and/or at least one potassium salt according to the present invention. For example, the additive may be a cathode material additive and/or an anode material additive and/or an electrolyte additive and/or a separator material additive.

In particular, the additive may be designed for an alkali metal cell and/or battery, in particular for an alkali metal-ion cell and/or battery, for example for a lithium cell and/or battery and/or for a sodium cell and/or battery, in particular for a lithium cell and/or battery, for example for a lithium-ion cell and/or battery and/or for a sodium-ion cell and/or battery, in particular for a lithium-ion cell and/or battery, for example in the form of a solid electrolyte cell and/or battery, for example in the form of a polymer electrolyte cell and/or battery, for example in the form of a solid-state cell and/or battery, such as in the form of a so-called all-solid-state cell and/or battery, for example having a high energy density and/or including a high-voltage cathode active material and/or having an elevated operating temperature.

With regard to further technical features and advantages of the additive according to the present invention, explicit reference is hereby made to the explanations in conjunction with the salt according to the present invention, the cell according to the present invention, the electrolyte according to the present invention, the battery according to the present invention, and the preparation method according to the present invention, and to the FIGURE and the description of the FIGURE.

Moreover, the present invention relates to an alkali metal cell, in particular an alkali metal-ion cell, for example a lithium cell and/or a sodium cell, in particular a lithium cell, for example a lithium-ion cell and/or a sodium-ion cell, in particular a lithium-ion cell, that includes at least one alkali metal salt, in particular lithium salt, according to the present invention, and/or at least one electrolyte according to the present invention and/or at least one additive according to the present invention.

The cell may be, for example, an alkali metal cell, in particular an alkali metal-ion cell, for example a lithium cell and/or a sodium cell, in particular a lithium cell, for example a lithium-ion cell and/or a sodium-ion cell, in particular a lithium-ion cell, for example in the form of a solid electrolyte cell, for example in the form of a polymer electrolyte cell, for example in the form of a solid-state cell, such as in the form of a so-called all-solid-state cell and/or battery, for example having a high energy density and/or including a high-voltage cathode active material and/or having an elevated operating temperature.

The cell may in particular include an anode, a cathode, and a separator situated between the anode and the cathode.

Within the scope of one specific embodiment, the cathode and/or the separator include(s) at least one electrolyte according to the present invention, in particular polymer electrolyte, and/or at least one additive according to the present invention.

Within the scope of another specific embodiment, the cathode includes at least one cathode active material having a cathode potential of ≥3.5 V, in particular ≥4.0 V, with respect to in particular metallic lithium (Li⁺/Li). For example, the at least one cathode active material may include or be lithium-nickel-cobalt-aluminum (NCA) oxide, for example LiNi_{0.8}Co_{0.15}Al_{0.05}O₅O₂ and/or lithium-nickel-manganese-cobalt (NMC) oxide(s), for example LiNi_{0.8}Mn_{0.1}Co_{0.1}O₂(NMC811), and/or at least one spinel compound, for example of the formula LiMₓMn₂₋ₓO₄, where M=Ni, Co, Cu, Cr, and/or Fe, for example LiMn₂O₄ and/or LiNi_{0.5}Mn_{1.5}O₄, and/or at least one olivine compound, for example of the formula LiMPO₄, where M=Mn, Ni, Co, Cu, Cr, and/or Fe, for example LiFePO₄ and/or LiMnPO₄.

Within the scope of another alternative or additional specific embodiment, the anode includes metallic lithium, in particular as anode active material. For example, the anode may be a lithium metal anode, for example in the form of a lithium metal foil.

With regard to further technical features and advantages of the cell according to the present invention, explicit reference is hereby made to the explanations in conjunction with the salt according to the present invention, the electrolyte according to the present invention, the additive according to the present invention, the battery according to the present invention, and the preparation method according to the present invention, and to the FIGURE and the description of the FIGURE.

Moreover, the present invention relates to an alkali metal battery, in particular an alkali metal-ion battery, for example a lithium battery and/or a sodium battery, in particular a lithium battery, for example a lithium-ion battery and/or a sodium-ion battery, in particular a lithium-ion battery, that include(s) at least one cell according to the present invention. In particular, the battery may include two or more cells according to the present invention.

The battery may be, for example, an alkali metal battery, in particular an alkali metal-ion battery, for example a lithium battery and/or a sodium battery, in particular a lithium battery, for example a lithium-ion battery and/or a sodium-ion battery, in particular a lithium-ion battery, for example in the form of a solid electrolyte battery, for example in the form of a polymer electrolyte battery, for example in the form of a solid-state battery, such as in the form of a so-called all-solid-state cell and/or battery, for example having a high energy density and/or including a high-voltage cathode active material and/or having an elevated operating temperature.

With regard to further technical features and advantages of the battery according to the present invention, explicit reference is hereby made to the explanations in conjunction with the salt according to the present invention, the electrolyte according to the present invention, the additive according to the present invention, the cell according to the present invention, and the preparation method according to the present invention, and to the FIGURE and the description of the FIGURE.

Moreover, the present invention relates to a method for preparing a salt according to the present invention.

In the method, a compound containing at least one (perfluoroalkyl)sulfonyl group, in particular S-(perfluoroalkyl)sulfonyl group, and containing at least one reactive group, in particular an electrophilic sulfur atom, is reacted with at least one unfluorinated, in particular unhalogenated, N,N-dialkylamine. It is thus advantageously possible to prepare a salt according to the present invention in the form of an ammonium compound salt, in particular with a dialkylammonium ion as cation, for example in high yields and/or high purity.

Subsequently, in particular directly afterward or after one or multiple reaction steps, a reaction with at least one alkali metal salt, for example a lithium salt, may be carried out.

The at least one alkali metal salt may include or be, for example, at least one lithium salt and/or at least one potassium salt and/or at least one sodium salt. In particular, the at least one alkali metal salt may include or be at least one lithium salt.

For example, the at least one alkali metal salt may include or be at least one alkali metal carbonate and/or at least one alkali metal hydroxide and/or at least one alkali metal nitride.

For example, the at least one alkali metal salt may include or be lithium carbonate (Li₂CO₃) and/or lithium hydroxide (LiOH) and/or lithium nitride (LiN₃) and/or potassium carbonate (K₂CO₃) and/or potassium hydroxide (KOH) and/or potassium nitride (KN₃) and/or sodium carbonate (Na₂CO₃) and/or sodium hydroxide (NaOH) and/or sodium nitride (NaN₃).

For example, an ammonium compound [(perfluoroalkyl)sulfonyl][(dialkylamide)sulfonyl]azanide, for example an ammonium compound-N-[(perfluoroalkyl)sulfonyl]-N-[(dialkylamide)sulfonyl]azanide, in particular with an ammonium compound, for example a dialkylammonium compound, as cation and with an imide-based anion, which in particular may be referred to as an azanide, for example a dialkylammonium [(perfluoroalkyl)sulfonyl][(dialkylamide)sulfonyl]azanide, may be prepared by reacting a [(perfluoroalkyl)sulfonyl][halosulfonyl]imide, for example an N-[(perfluoroalkyl)sulfonyl]-N-[halosulfonyl]imide, with at least one unfluorinated, for example unhalogenated, N,N-dialkylamine, in particular according to the following reaction scheme 1:

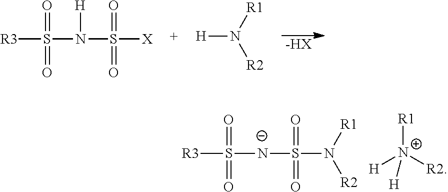

R1, R2, and R3 may in particular be as explained in conjunction with the salt according to the present invention. X may in particular stand for a halogen, for example chlorine.

Thus, for example, methylethylammonium [(trifluoromethyl)sulfonyl][(methylethylamide)sulfonyl]azanide may be prepared by reacting [(trifluoromethyl)sulfonyl][chlorosulfonyl]imide, for example N-[(trifluoromethyl)sulfonyl]-N-[chlorosulfonyl]imide, with methylethylamine according to the following reaction scheme 1′:

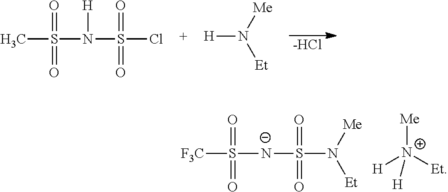

Analogously, for example dimethylammonium [(trifluoromethyl)sulfonyl][(dimethylamide)sulfonyl]azanide may be prepared by reacting [(trifluoromethyl)sulfonyl][chlorosulfonyl]imide, for example N-[(trifluoromethyl)sulfonyl]-N-[chlorosulfonyl]imide, with dimethylamine, or diethylammonium [(trifluoromethyl)sulfonyl][(diethylamide)sulfonyl]azanide may be prepared by reacting [(trifluoromethyl)sulfonyl][chlorosulfonyl]imide, for example N-[(trifluoromethyl)sulfonyl]-N-[chlorosulfonyl]imide, with diethylamine.

The ammonium compound [(perfluoroalkyl)sulfonyl][(dialkylamide)sulfonyl]azanide, for example the ammonium compound-N-[(perfluoroalkyl)sulfonyl]-N-[(dialkylamide)sulfonyl]azanide, in particular the reaction product from reaction scheme 1, or methylethylammonium [(trifluoromethyl)sulfonyl][(methylethylamide)sulfonyl]azanide, in particular the reaction product from reaction scheme 1′, or the dimethylammonium [(trifluoromethyl)sulfonyl][(dimethylamide)sulfonyl]azanide or diethylammonium[(trifluoromethyl)sulfonyl][(diethylamide)sulfonyl]azanide prepared in particular analogously thereto, may be prepared by reacting with at least one alkali metal salt, for example an alkali metal carbonate and/or alkali metal hydroxide and/or alkali metal nitride, for example with at least one lithium salt, for example lithium carbonate (Li₂CO₃) and/or lithium hydroxide (LiOH) and/or lithium nitride (LiN₃), or with at least one potassium salt, for example potassium carbonate (K₂CO₃) and/or potassium hydroxide (KOH) and/or potassium nitride (KN₃), or with at least one sodium salt, for example sodium carbonate (Na₂CO₃) and/or sodium hydroxide (NaOH) and/or sodium nitride (NaN₃), to give an alkali metal [(perfluoroalkyl)sulfonyl][(dialkylamide)sulfonyl]azanide, for example an alkali metal N-[(perfluoroalkyl)sulfonyl]-N-[(dialkylamide)sulfonyl]azanide, or an alkali metal [(trifluoromethyl)sulfonyl][(methylethylamide)sulfonyl]azanide or an alkali metal [(trifluoromethyl)sulfonyl][(dimethylamide)sulfonyl]azanide or an alkali metal [(trifluoromethyl)sulfonyl][(diethylamide)sulfonyl]azanide. It is thus advantageously possible to prepare a salt according to the present invention in the form of an alkali metal salt, for example in the form of a lithium salt or sodium salt or potassium salt.

It is possible to carry out the reaction with the at least one unfluorinated, in particular unhalogenated, N,N-dialkylamine and the reaction with the at least one alkali metal salt in succession and/or in a reaction vessel, in particular as a so-called one-pot reaction. The alkali metal salt may thus be prepared in a particularly simple and cost-effective manner.

For example, the reaction product of the reaction with the at least one unfluorinated, in particular unhalogenated, N,N-dialkylamine, in particular from reaction scheme 1 or 1′, for example the ammonium compound [(perfluoroalkyl)sulfonyl][(dialkylamide)sulfonyl]azanide, for example the ammonium compound-N-[(perfluoroalkyl)sulfonyl]-N-[(dialkylamide)sulfonyl]azanide, or the methylethylammonium[(trifluoromethyl)sulfonyl][(methylethylamide)sulfonyl]azanide, or the dimethylammonium [(trifluoromethyl)sulfonyl][(dimethylamide)sulfonyl]azanide or diethylammonium [(trifluoromethyl)sulfonyl][(diethylamide)sulfonyl]azanide prepared in particular analogously thereto, may be reacted in direct succession, in particular in the reaction shown in reaction scheme 1 or 1′, with at least one alkali metal salt, in particular according to the following reaction scheme 2*:

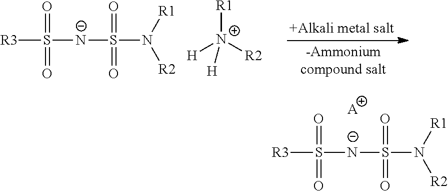

in particular to give an alkali metal [(perfluoroalkyl)sulfonyl][(dialkylamide)sulfonyl]azanide, for example an alkali metal N-[(perfluoroalkyl)sulfonyl]-N-[(dialkylamide)sulfonyl]azanide, or an alkali metal [(trifluoromethyl)sulfonyl][(methylethylamide)sulfonyl]azanide or an alkali metal [(trifluoromethyl)sulfonyl][(dimethylamide)sulfonyl]azanide or an alkali metal [(trifluoromethyl)sulfonyl][(diethylamide)sulfonyl]azanide. R1, R2, and R3 may in particular be as explained in conjunction with the salt according to the present invention. A⁺ may in particular stand for an alkali metal ion, for example a lithium ion (Li⁺) or a sodium ion (Na⁺) or a potassium ion (K+), in particular a lithium ion (Li⁺). The at least one alkali metal salt may in particular include or be at least one alkali metal hydroxide, for example lithium hydroxide (LiOH) and/or potassium hydroxide (KOH) and/or sodium hydroxide (NaOH). The ammonium compound salt that is formed may in particular include or be formed from the ammonium compound, for example the dialkylammonium compound, for example the NH₂R1R2 ammonium ion, as cation, and the anion of the alkali metal salt that is used, for example a hydroxide anion.

Lithium [(trifluoromethyl)sulfonyl][(methylethylamide)sulfonyl]azanide may be prepared in such a way, for example, that the reaction product of the reaction with the at least one unfluorinated, in particular unhalogenated, N,N-dialkylamine, for example methylethylamine, in particular from reaction scheme 1′, for example the methylethylammonium [(trifluoromethyl)sulfonyl][(methylethylamide)sulfonyl]azanide, is reacted in direct succession, in particular in the reaction shown in reaction scheme 1′, with lithium hydroxide, in particular according to the following particular reaction scheme (2*′):

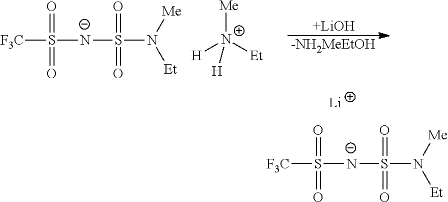

Analogously, for example lithium [(trifluoromethyl)sulfonyl][(dimethylamide)sulfonyl]azanide may be prepared by reacting the reaction product of the reaction with the at least one unfluorinated, in particular unhalogenated, N,N-dialkylamine, for example dimethylamine, in particular the dimethylammonium [(trifluoromethyl)sulfonyl][(dimethylamide)sulfonyl]azanide, in direct succession with lithium hydroxide. Lithium [(trifluoromethyl)sulfonyl][(diethylamide)sulfonyl]azanide may be analogously prepared, for example, by reacting the reaction product of the reaction with the at least one unfluorinated, in particular unhalogenated, N,N-dialkylamine, for example diethylamine, in particular diethylammonium [(trifluoromethyl)sulfonyl][(diethylamide)sulfonyl]azanide, in direct succession with lithium hydroxide.

Potassium [(trifluoromethyl)sulfonyl][(methylethylamide)sulfonyl]azanide may be prepared in such a way that, for example, the reaction product of the reaction with the at least one unfluorinated, in particular unhalogenated, N,N-dialkylamine, for example methylethylamine, in particular from reaction scheme 1′, for example the methylethylammonium [(trifluoromethyl)sulfonyl][(methylethylamide)sulfonyl]azanide, is reacted in direct succession, in particular in the reaction shown in reaction scheme 1′, with potassium hydroxide, in particular according to the following particular reaction scheme 2*″:

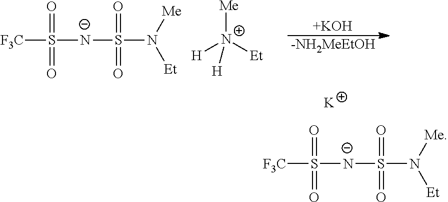

Analogously, for example potassium [(trifluoromethyl)sulfonyl][(dimethylamide)sulfonyl]azanide may be prepared by reacting the reaction product of the reaction with the at least one unfluorinated, in particular unhalogenated, N,N-dialkylamine, for example dimethylamine, in particular the dimethylammonium [(trifluoromethyl)sulfonyl][(dimethylamide)sulfonyl]azanide, in direct succession with potassium hydroxide. Potassium [(trifluoromethyl)sulfonyl][(diethylamide)sulfonyl]azanide may be analogously prepared, for example, by reacting the reaction product of the reaction with the at least one unfluorinated, in particular unhalogenated, N,N-dialkylamine, for example diethylamine, in particular the diethylammonium [(trifluoromethyl)sulfonyl][(diethylamide)sulfonyl]azanide, in direct succession with potassium hydroxide.

However, to increase the purity and avoid product workup, for example, it may be advantageous to carry out the preparation method as a multistep synthesis method.

For example, after the reaction with the at least one unfluorinated, in particular unhalogenated, N,N-dialkylamine, for example methylethylamine or dimethylamine or diethylamine, in particular according to reaction scheme 1 or 1′, the reaction product thereof, in particular the ammonium compound [(perfluoroalkyl)sulfonyl][(dialkylamide)sulfonyl]azanide, for example the ammonium compound-N-[(perfluoroalkyl)sulfonyl]-N-[(dialkylamide)sulfonyl]azanide, or the methylethylammonium[(trifluoromethyl)sulfonyl][(methylethylamide)sulfonyl]azanide, or the dimethylammonium [(trifluoromethyl)sulfonyl][(dimethylamide)sulfonyl]azanide or the diethylammonium [(trifluoromethyl)sulfonyl][(diethylamide)sulfonyl]azanide, is first neutralized by a reaction, in particular following the reaction shown in reaction scheme 1 or 1′, that is carried out with at least one hydrogen acid (HX), for example at least one hydrogen halide, in particular according to the following reaction scheme 2:

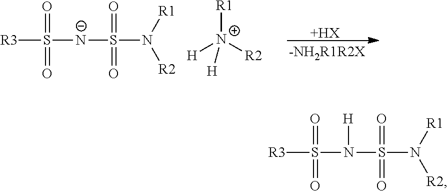

in particular to give a hydrogen [(perfluoroalkyl)sulfonyl][(dialkylamide)sulfonyl]amine, for example a hydrogen-N-[(perfluoroalkyl)sulfonyl]-N-[(dialkylamide)sulfonyl]amine, or hydrogen [(trifluoromethyl)sulfonyl][(methylethylamide)sulfonyl]amine or hydrogen [(trifluoromethyl)sulfonyl][(dimethylamide)sulfonyl]amine or hydrogen [(trifluoromethyl)sulfonyl][(diethylamide)sulfonyl]amine. R1, R2, and R3 may in particular be as explained in conjunction with the salt according to the present invention. X may in particular stand for a halogen, for example chlorine.

Hydrogen [(trifluoromethyl)sulfonyl][(methylethylamide)sulfonyl]amine may be prepared in such a way, for example, that the reaction product of the reaction with the at least one unfluorinated, in particular unhalogenated, N,N-dialkylamine, for example methylethylamine, in particular from reaction scheme 1′, for example the methylethylammonium [(trifluoromethyl)sulfonyl][(methylethylamide)sulfonyl]azanide, is first neutralized with hydrochloric acid (hydrogen chloride), in particular according to the following particular reaction scheme 2′:

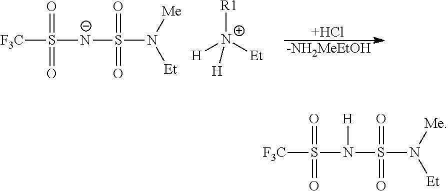

Analogously, for example hydrogen [(trifluoromethyl)sulfonyl][(dimethylamide)sulfonyl]amine may be prepared by first neutralizing the reaction product of the reaction with the at least one unfluorinated, in particular unhalogenated, N,N-dialkylamine, for example dimethylamine, for example the dimethylammonium [(trifluoromethyl)sulfonyl][(dimethylamide)sulfonyl]azanide, with hydrochloric acid (hydrogen chloride). Hydrogen [(trifluoromethyl)sulfonyl][(diethylamide)sulfonyl]amine may be analogously prepared, for example, by first neutralizing the reaction product of the reaction with the at least one unfluorinated, in particular unhalogenated, N,N-dialkylamine, for example diethylamine, for example diethylammonium [(trifluoromethyl)sulfonyl][(diethylamide)sulfonyl]azanide, with hydrochloric acid (hydrogen chloride).

After the neutralization, in particular according to reaction scheme 2 or 2′, the reaction product thereof, in particular the hydrogen [(perfluoroalkyl)sulfonyl][(dialkylamide)sulfonyl]amine, for example the hydrogen-N-[(perfluoroalkyl)sulfonyl]-N-[(dialkylamide)sulfonyl]amine, or the hydrogen [(trifluoromethyl)sulfonyl][(methylethylamide)sulfonyl]amine or the hydrogen [(trifluoromethyl)sulfonyl][(dimethylamide)sulfonyl]amine or the hydrogen [(trifluoromethyl)sulfonyl][(diethylamide)sulfonyl]amine, may then be reacted with at least one alkali metal salt, in particular according to reaction scheme 3:

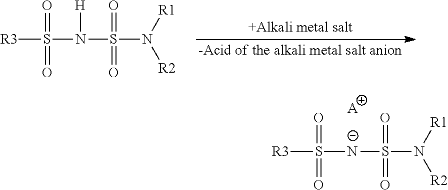

in particular to give an alkali metal [(perfluoroalkyl)sulfonyl][(dialkylamide)sulfonyl]azanide, for example an alkali metal N-[(perfluoroalkyl)sulfonyl]-N-[(dialkylamide)sulfonyl]azanide, or an alkali metal [(trifluoromethyl)sulfonyl][(methylethylamide)sulfonyl]azanide or an alkali metal [(trifluoromethyl)sulfonyl][(dimethylamide)sulfonyl]azanide or an alkali metal [(trifluoromethyl)sulfonyl][(diethylamide)sulfonyl]azanide. R1, R2, and R3 may in particular be as explained in conjunction with the salt according to the present invention. A⁺ may in particular stand for an alkali metal ion, for example a lithium ion (Li⁺) or a potassium ion (K⁺) or a sodium ion (Na⁺), in particular a lithium ion (Li⁺). The at least one alkali metal salt may include or be in particular at least one alkali metal carbonate, for example lithium carbonate (Li₂CO₃) and/or potassium carbonate (K₂CO₃) and/or sodium carbonate (Na₂CO₃). The acid of the alkali metal salt anion formed in the reaction scheme may be carbonic acid (H₂CO₃), for example.

Lithium [(trifluoromethyl)sulfonyl][(methylethylamide)sulfonyl]azanide may be prepared, for example, by reacting the reaction product of the neutralization, in particular from reaction scheme 2′, for example the hydrogen [(trifluoromethyl)sulfonyl][(methylethylamide)sulfonyl]amine, with at least one lithium salt, for example lithium carbonate, in particular according to the following particular reaction scheme 3′:

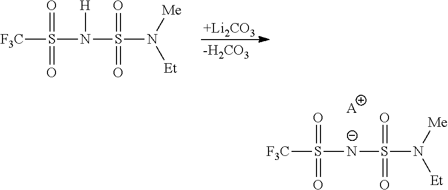

Analogously, for example lithium [(trifluoromethyl)sulfonyl][(dimethylamide)sulfonyl]azanide may be prepared by reacting the reaction product of the neutralization, for example the hydrogen [(trifluoromethyl)sulfonyl][(dimethylamide)sulfonyl]amine, with at least one lithium salt, for example lithium carbonate.

Lithium [(trifluoromethyl)sulfonyl][(diethylamide)sulfonyl]azanide may be analogously prepared, for example, by reacting the reaction product of the neutralization, for example the hydrogen [(trifluoromethyl)sulfonyl][(diethyl-amide)sulfonyl]amine, with at least one lithium salt, for example lithium carbonate.

Potassium [(trifluoromethyl)sulfonyl][(methylethylamide)sulfonyl]azanide may be prepared, for example, by reacting the reaction product of the neutralization, in particular from reaction scheme 2′, for example the hydrogen [(trifluoromethyl)sulfonyl][(methylethylamide)sulfonyl]amine, with at least one potassium salt, for example potassium carbonate, in particular according to the following particular reaction scheme 3″:

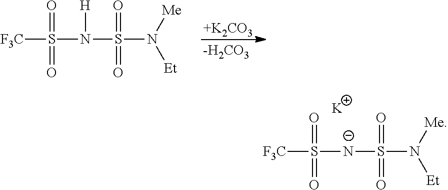

Analogously, for example potassium [(trifluoromethyl)sulfonyl][(dimethylamide)sulfonyl]azanide may be prepared by reacting the reaction product of the neutralization, for example the hydrogen [(trifluoromethyl)sulfonyl][(dimethylamide)sulfonyl]amine, with at least one potassium salt, for example potassium carbonate.

Potassium [(trifluoromethyl)sulfonyl][(diethylamide)sulfonyl]azanide may be analogously prepared, for example, by reacting the reaction product of the neutralization, for example the hydrogen [(trifluoromethyl)sulfonyl][(diethylamide)sulfonyl]amine, with at least one potassium salt, for example potassium carbonate.

A salt whose anion contains at least one (N,N-dialkylamide)sulfoximide group and at least one (perfluoroalkyl)sulfonyl group, for example at least two (N,N-dialkylamide)sulfoximide groups and at least two (perfluoroalkyl)sulfonyl groups, for example a salt of the general chemical formula:

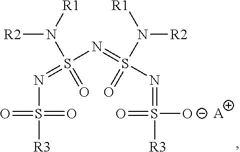

may be prepared, for example, by reacting at least one [(perfluoroalkyl)sulfonyl]sulfoximide, for example N-[(perfluoroalkyl)sulfonyl]sulfoximide, with at least one unfluorinated, in particular unhalogenated, N,N-dialkylamine, for example via the synthesis routes described below.

For example, the [(perfluoroalkyl)sulfonyl]sulfoximide, for example the N-[(perfluoroalkyl)sulfonyl]sulfoximide, may be prepared by first reacting a [(perfluoroalkyl)sulfonyl]sulfonamide with for example thionyl chloride (dichlorosulfoxide), for example according to the following reaction scheme I:

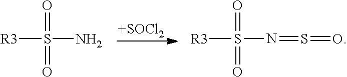

R1, R2, and R3 may in particular be as explained in conjunction with the salt according to the present invention.

For example, [(trifluoromethyl)sulfonyl]sulfoximide, for example N-[(trifluoromethyl)sulfonyl]sulfoximide, may be prepared by reacting [(trifluoromethyl)sulfonyl]sulfonamide with for example thionyl chloride (dichlorosulfoxide), for example according to the following reaction scheme I′:

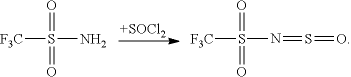

Within the scope of one embodiment, the [(perfluoroalkyl)sulfonyl]sulfoximide, for example the N-[(perfluoroalkyl)sulfonyl]sulfoximide, in particular the reaction product from reaction scheme I, may be reacted with the at least one unfluorinated, in particular unhalogenated, N,N-dialkylamine in an organic amine as solvent (amine solvent), in particular according to reaction scheme IIa:

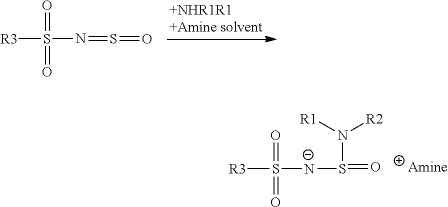

in particular to give an ammonium compound [(perfluoroalkyl)sulfonyl][dialkylamide]sulfoximide salt, for example an ammonium compound-N-[(perfluoroalkyl)sulfonyl]-S-[dialkylamide]sulfoximide salt, in particular reacted with an ammonium compound (Amine⁺) as cation and with a sulfoximide-based anion. R1, R2, and R3 may also in particular be as explained in conjunction with the salt according to the present invention.

For example, an ammonium compound [(trifluoromethyl)sulfonyl][methylethylamide]sulfoximide salt, for example an ammonium compound-N-[(trifluoromethyl)sulfonyl]-S-[methylethylamide]sulfoximide salt, may be prepared by reacting [(trifluoromethyl)sulfonyl]sulfoximide, for example N-[(trifluoromethyl)sulfonyl]sulfoximide, with methylethylamine in at least one amine, for example triethylamine and/or diisopropylethylamine (DIPEA), as solvent, for example according to the following reaction scheme IIa′:

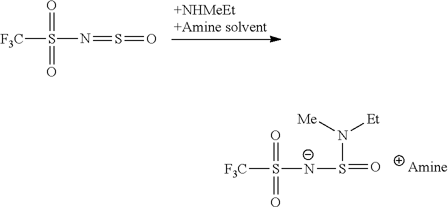 [SMILES: O=S=NS(=O)(=O)C(F)(F)F ; CCN(C)S(=O)[N-]S(=O)(=O)C(F)(F)F]

Analogously, for example an ammonium compound [(trifluoromethyl)sulfonyl][dimethylamide]sulfoximide salt, for example an ammonium compound-N-[(trifluoromethyl)sulfonyl]-S-[dimethylamide]sulfoximide salt, may be prepared by reacting [(trifluoromethyl)sulfonyl]sulfoximide, for example N-[(trifluoromethyl)sulfonyl]sulfoximide, with dimethylamine in at least one amine, for example triethylamine and/or diisopropylethylamine (DIPEA), as solvent. An ammonium compound [(trifluoromethyl)sulfonyl][diethylamide]sulfoximide salt, for example an ammonium compound-N-[(trifluoromethyl)sulfonyl]-S-[diethylamide]sulfoximide salt, may be analogously prepared, for example, by reacting [(trifluoromethyl)sulfonyl]sulfoximide, for example N-[(trifluoromethyl)sulfonyl]sulfoximide, with diethylamine in at least one amine, for example triethylamine and/or diisopropylethylamine (DIPEA), as solvent.

Within the scope of another embodiment, the [(perfluoroalkyl)sulfonyl]sulfoximide, for example the N-[(perfluoroalkyl)sulfonyl]sulfoximide, in particular the reaction product from reaction scheme I, may be reacted first with cesium fluoride and then with the at least one unfluorinated, in particular unhalogenated, N,N-dialkylamine, in particular according to reaction scheme IIb:

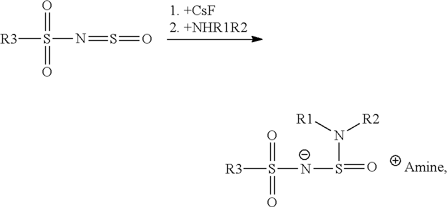

in particular to give an ammonium compound [(perfluoroalkyl)sulfonyl][dialkylamide]sulfoximide salt, for example an ammonium compound-N-(perfluoroalkyl)sulfonyl]-S-[dialkylamide]sulfoximide salt, in particular with an ammonium compound (Amine⁺) as cation and with a sulfoximide-based anion. R1, R2, and R3 may also in particular be as explained in conjunction with the salt according to the present invention.

For example, an ammonium compound [(trifluoromethyl)sulfonyl][methylethylamide]sulfoximide salt, for example an ammonium compound-N-[(trifluoromethyl)sulfonyl]-S-[methylethylamide]sulfoximide salt, may be prepared by reacting [(trifluoromethyl)sulfonyl]sulfoximide, for example N-[(trifluoromethyl)sulfonyl]sulfoximide, first with cesium fluoride and then with methylethylamine, for example according to the following reaction scheme IIb′:

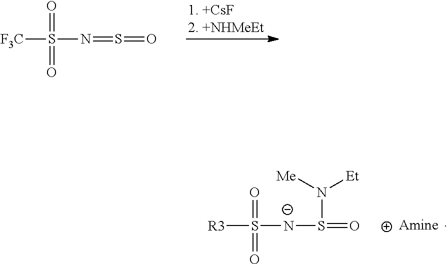

Analogously, for example an ammonium compound [(trifluoromethyl)sulfonyl][dimethylamide]sulfoximide salt, for example an ammonium compound-N-[(trifluoromethyl)sulfonyl]-S-[dimethylamide]sulfoximide salt, may be prepared by reacting [(trifluoromethyl)sulfonyl]sulfoximide, for example N-[(trifluoromethyl)sulfonyl]sulfoximide, first with cesium fluoride and then with dimethylamine. An ammonium compound [(trifluoromethyl)sulfonyl][diethylamide]sulfoximide salt, for example an ammonium compound-N-[(trifluoromethyl)sulfonyl]-S-[diethylamide]sulfoximide salt, may be analogously prepared, for example, by reacting [(trifluoromethyl)sulfonyl]sulfoximide, for example N-[(trifluoromethyl)sulfonyl]sulfoximide, first with cesium fluoride and then with diethylamine.

After the reaction with the at least one unfluorinated, in particular unhalogenated, N,N-dialkylamine, in particular according to reaction scheme IIa or IIb, the reaction product thereof, in particular the ammonium compound [(perfluoroalkyl)sulfonyl][dialkylamide]sulfoximide salt, for example the ammonium compound-N-[(perfluoroalkyl)sulfonyl]-S-[dialkylamide]sulfoximide salt, may then be reacted in particular with an ammonium compound (Amine⁺) as cation and with a sulfoximide-based anion, using at least one oxidation agent and fluorination agent, for example using a combination of elemental chlorine (Cl₂) and cesium fluoride (CsF), in particular according to reaction scheme III:

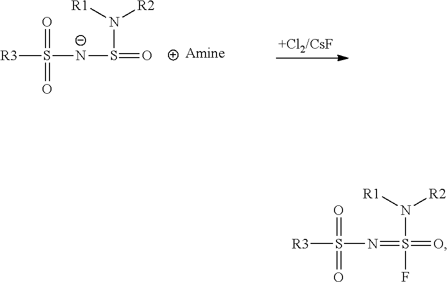

in particular to give a [(perfluoroalkyl)sulfonyl][dialkylamide]fluorosulfoximide, for example an N-[(perfluoroalkyl)sulfonyl]-S-[dialkylamide]-S-fluorosulfoximide. R1, R2, and R3 may also in particular be as explained in conjunction with the salt according to the present invention.

For example, a [(trifluoromethyl)sulfonyl][methylethylamide]fluorosulfoximide, for example an N-[(trifluoromethyl)sulfonyl]-S-[methylethylamide]-S-fluorosulfoximide, may be prepared by reacting an ammonium compound [(trifluoromethyl)sulfonyl][methylethylamide]sulfoximide salt, for example an ammonium compound-N-[(trifluoromethyl)sulfonyl]-S-[methylethylamide]sulfoximide salt, with at least one oxidation agent and fluorination agent, for example in the form of a combination of elemental chlorine (Cl₂) and cesium fluoride (CsF), for example according to the following reaction scheme III′:

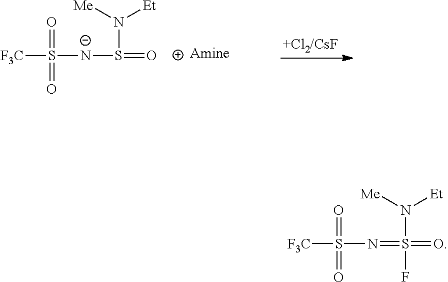 [SMILES: CCN(C)S(=O)[N-]S(=O)(=O)C(F)(F)F ; CCN(C)S(=O)(F)=NS(=O)(=O)C(F)(F)F]

Analogously, for example a [(trifluoromethyl)sulfonyl][dimethylamide]fluorosulfoximide, for example an N-[(trifluoromethyl)sulfonyl]-S-[dimethylamide]-S-fluorosulfoximide, may be prepared by reacting an ammonium compound [(trifluoromethyl)sulfonyl][dimethylamide]sulfoximide salt, for example an ammonium compound-N-[(trifluoromethyl)sulfonyl]-S-[dimethylamide]sulfoximide salt, with at least one oxidation agent and fluorination agent, for example in the form of a combination of elemental chlorine (Cl₂) and cesium fluoride (CsF). A [(trifluoromethyl)sulfonyl][diethylamide]fluorosulfoximide, for example an N-[(trifluoromethyl)sulfonyl]-S-[diethylamide]-S-fluorosulfoximide, may be analogously prepared, for example, by reacting an ammonium compound [(trifluoromethyl)sulfonyl][diethylamide]sulfoximide salt, for example an ammonium compound-N-[(trifluoromethyl)sulfonyl]-S-[diethylamide]sulfoximide salt, with at least one oxidation agent and fluorination agent, for example in the form of a combination of elemental chlorine (Cl₂) and cesium fluoride (CsF).

Within the scope of one embodiment, after the reaction with the at least one oxidation agent and fluorination agent, in particular according to reaction scheme III, the reaction product thereof, in particular the [(perfluoroalkyl)sulfonyl][dialkylamide]fluorosulfoximide, for example the N-[(perfluoroalkyl)sulfonyl]-S-[dialkylamide]-S-fluorosulfoximide, may be reacted with at least one alkali metal salt, for example at least one alkali metal nitride, in particular according to reaction scheme IVa:

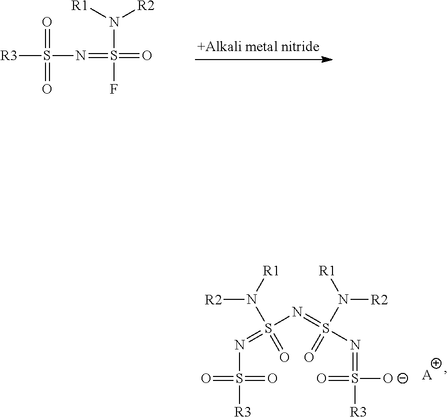

in particular to give an alkali metal salt whose anion contains at least one (N,N-dialkylamide)sulfoximide group and at least one (perfluoroalkyl)sulfonyl group, in particular at least two (N,N-dialkylamide)sulfoximide groups and at least two (perfluoroalkyl)sulfonyl groups, for example a salt of the general chemical formula:

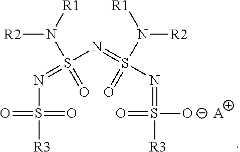

R1, R2, and R3 may also in particular be as explained in conjunction with the salt according to the present invention.

For example, a lithium salt whose anion contains at least one (N,N-methylethylamide)sulfoximide group and at least one (trifluoromethyl)sulfonyl group, in particular at least two (N,N-methylethylamide)sulfoximide groups and at least two (trifluoromethyl)sulfonyl groups, may be prepared, for example, to give a salt of the general chemical formula:

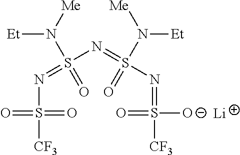 [SMILES: CCN(C)S(=O)(=NS(=O)(=O)C(F)(F)F)N=S(=O)(N=S(=O)([O-])C(F)(F)F)N(C)CC ; [Li+]]

by reacting the [(trifluoromethyl)sulfonyl][methylethylamide]fluorosulfoximide, for example the N-[(trifluoromethyl)sulfonyl]-S-[methylethylamide]-S-fluorosulfoximide, with at least one lithium salt, for example lithium nitride (LiN₃), for example according to the following reaction scheme IVa′:

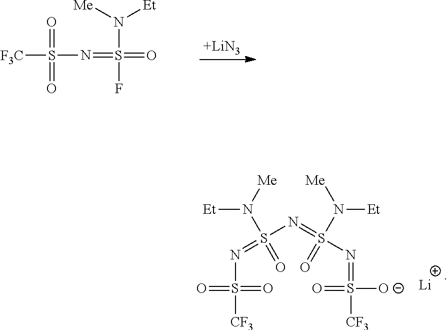 [SMILES: "+LiN3" ; CCN(C)S(=O)(F)=NS(=O)(=O)C(F)(F)F ; CCN(C)S(=O)(=NS(=O)(=O)C(F)(F)F)N=S(=O)(N=S(=O)([O-])C(F)(F)F)N(C)CC ; [Li+]]

Analogously, for example a lithium salt whose anion contains at least one (N,N-dimethylamide)sulfoximide group and at least one (trifluoromethyl)sulfonyl group, in particular at least two (N,N-dimethylamide)sulfoximide groups and at least two (trifluoromethyl)sulfonyl groups, may be prepared, for example, to give a salt of the general chemical formula:

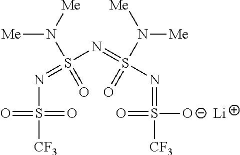 [SMILES: CN(C)S(=O)(=NS(=O)(=O)C(F)(F)F)N=S(=O)(N=S(=O)([O-])C(F)(F)F)N(C)C ; [Li+]]

by reacting the [(trifluoromethyl)sulfonyl][dimethylamide]fluorosulfoximide, for example the N-[(trifluoromethyl)sulfonyl]-S-[dimethylamide]-S-fluorosulfoximide, with at least one lithium salt, for example lithium nitride (LiN₃).

A lithium salt whose anion contains at least one (N,N-diethylamide)sulfoximide group and at least one (trifluoromethyl)sulfonyl group, in particular at least two (N,N-diethylamide)sulfoximide groups and at least two (trifluoromethyl)sulfonyl groups, for example to give a salt of the general chemical formula:

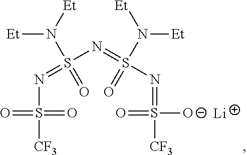 [SMILES: CCN(CC)S(=O)(=NS(=O)(=O)C(F)(F)F)N=S(=O)(N=S(=O)([O-])C(F)(F)F)N(CC)CC ; [Li+]]

may be analogously prepared, for example, by reacting the [(trifluoromethyl)sulfonyl][diethylamide]fluorosulfoximide, for example the N-[(trifluoromethyl)sulfonyl]-S-[diethylamide]-S-fluorosulfoximide, with at least one lithium salt, for example lithium nitride (LiN₃).

Within the scope of another embodiment, after the reaction with the at least one oxidation agent and fluorination agent, in particular according to reaction scheme III, the reaction product thereof, in particular the [(perfluoroalkyl)sulfonyl][dialkylamide]fluorosulfoximide, for example the N-[(perfluoroalkyl)sulfonyl]-S-[dialkylamide]-S-fluorosulfoximide, may be reacted with at least one amination agent, for example an ammonium salt of an amine-substituted carboxylic acid, for example the ammonium salt of amine-substituted formic acid (NH₄O₂CNH₂), in particular according to reaction scheme IVb:

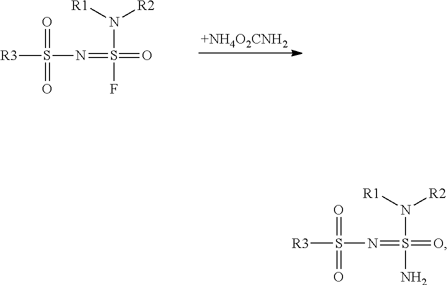

in particular to give a [(perfluoroalkyl)sulfonyl][dialkylamide]aminosulfoximide, for example an N-[(perfluoroalkyl)sulfonyl]-S-[dialkylamide]-S-aminosulfoximide. R1, R2, and R3 may also in particular be as explained in conjunction with the salt according to the present invention,

For example, a [(trifluoromethyl)sulfonyl][methylethylamide]aminosulfoximide, for example an N-[(trifluoromethyl)sulfonyl]-S-[methylethylamide]-S-aminosulfoximide, may be prepared by reacting the [(trifluoromethyl)sulfonyl][methylethylamide]fluorosulfoximide salt, for example the N-[(trifluoromethyl)sulfonyl]-S-[methylethylamide]-S-fluorosulfoximide salt, in particular from reaction scheme III′, with at least one amination agent, for example the ammonium salt of amine-substituted formic acid (NH₄O₂CNH₂), for example according to the following reaction scheme IVb′:

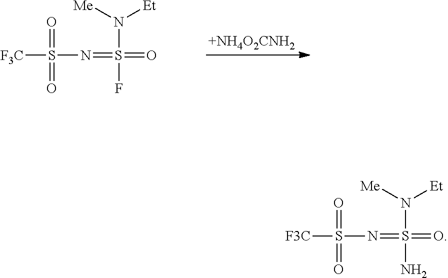 [SMILES: CCN(C)S(=O)(F)=NS(=O)(=O)C(F)(F)F ; "+NH4O2CNH2" ; CCN(C)S(N)(=O)=NS(=O)(=O)C(F)(F)F]

After the reaction with the at least one amination agent, in particular according to reaction scheme IVb, the reaction product thereof, in particular the [(perfluoroalkyl)sulfonyl][dialkylamide]aminosulfoximide, for example the N-[(perfluoroalkyl)sulfonyl]-S-[dialkylamide]-S-aminosulfoximide, may be reacted with a further reaction product from the reaction with at least one oxidation agent and fluorination agent, in particular according to reaction scheme III, for example with [(perfluoroalkyl)sulfonyl][dialkylamide]fluorosulfoximide, for example N-[(perfluoroalkyl)sulfonyl]-S-[dialkylamide]-S-fluorosulfoximide, and with at least one alkali metal salt, for example at least one alkali metal hydroxide, in particular according to reaction scheme IVb′:

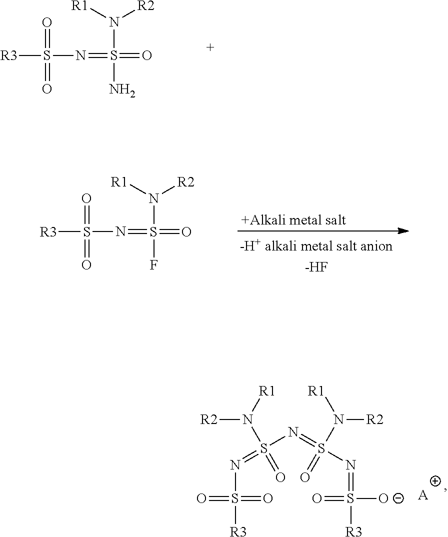

in particular to give an alkali metal salt whose anion contains at least one (N,N-dialkylamide)sulfoximide group and at least one (perfluoroalkyl)sulfonyl group, in particular at least two (N,N-dialkylamide)sulfoximide groups and at least two (perfluoroalkyl)sulfonyl groups, for example to give a salt of the general chemical formula:

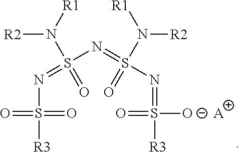

R1, R2, and R3 may also in particular be as explained in conjunction with the salt according to the present invention.

For example, a lithium salt whose anion contains at least one (N,N-methylethylamide)sulfoximide group and at least one (trifluoromethyl)sulfonyl group, in particular at least two (N,N-methylethylamide)sulfoximide groups and at least two (trifluoromethyl)sulfonyl groups, may be prepared, for example, to give a salt of the general chemical formula:

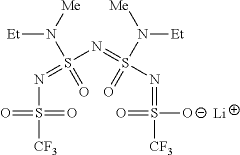 [SMILES: CCN(C)S(=O)(=NS(=O)(=O)C(F)(F)F)N=S(=O)(N=S(=O)([O-])C(F)(F)F)N(C)CC ; [Li+]]

by reacting the [(trifluoromethyl)sulfonyl][methylethylamide]aminosulfoximide, for example the N-[(trifluoromethyl)sulfonyl]-S-[methylethylamide]-S-aminosulfoximide, in particular from reaction scheme IVb, with a further reaction product from the reaction with at least one oxidation agent and fluorination agent, in particular according to reaction scheme III′, for example with the [(trifluoromethyl)sulfonyl][methylethylamide]fluorosulfoximide, for example the N-[(trifluoromethyl)sulfonyl]-S-[methylethylamide]-S-fluorosulfoximide, and with at least one lithium salt, for example lithium hydroxide, for example according to the following reaction scheme IVb″:

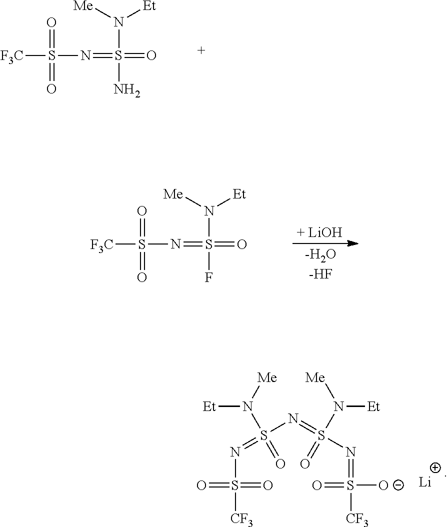 [SMILES: CCN(C)S(N)(=O)=NS(=O)(=O)C(F)(F)F ; CCN(C)S(=O)(F)=NS(=O)(=O)C(F)(F)F ; "+ LiOH" ; CCN(C)S(=O)(=NS(=O)(=O)C(F)(F)F)N=S(=O)(N=S(=O)([O-])C(F)(F)F)N(C)CC ; [Li+]]

Analogously, for example a lithium salt whose anion contains at least one (N,N-dimethylamide)sulfoximide group and at least one (trifluoromethyl)sulfonyl group, in particular at least two (N,N-dimethylamide)sulfoximide groups and at least two (trifluoromethyl)sulfonyl groups, may be prepared, for example, to give a salt of the general chemical formula:

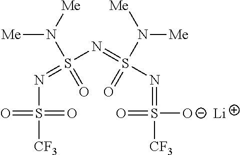 [SMILES: CN(C)S(=O)(=NS(=O)(=O)C(F)(F)F)N=S(=O)(N=S(=O)([O-])C(F)(F)F)N(C)C ; [Li+]]

by reacting the [(trifluoromethyl)sulfonyl][dimethylamide]aminosulfoximide, for example the N-[(trifluoromethyl)sulfonyl]-S-[dimethylamide]-S-aminosulfoximide, with a further reaction product from the reaction with at least one oxidation agent and fluorination agent, for example with the [(trifluoromethyl)sulfonyl][dimethylamide]fluorosulfoximide, for example the N-[(trifluoromethyl)sulfonyl]-S-[dimethylamide]-S-fluorosulfoximide, and with at least one lithium salt, for example lithium hydroxide.

A lithium salt whose anion contains at least one (N,N-diethylamide)sulfoximide group and at least one (trifluoromethyl)sulfonyl group, in particular at least two (N,N-diethylamide)sulfoximide groups and at least two (trifluoromethyl)sulfonyl groups, for example to give a salt of the general chemical formula:

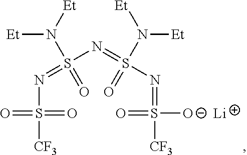 [SMILES: CCN(CC)S(=O)(=NS(=O)(=O)C(F)(F)F)N=S(=O)(N=S(=O)([O-])C(F)(F)F)N(CC)CC ; [Li+]]

may be analogously prepared, for example, by reacting the [(trifluoromethyl)sulfonyl][diethylamide]aminosulfoximide, for example the N-[(trifluoromethyl)sulfonyl]-S-[diethylamide]-S-aminosulfoximide, with a further reaction product from the reaction with at least one oxidation agent and fluorination agent, for example with the [(trifluoromethyl)sulfonyl][diethylamide]-fluorosulfoximide, for example the N-[(trifluoromethyl)sulfonyl]-S-[diethylamide]-S-fluorosulfoximide, and with at least one lithium salt, for example lithium hydroxide.

With regard to further technical features and advantages of the preparation method according to the present invention, explicit reference is hereby made to the explanations in conjunction with the salt according to the present invention, the electrolyte according to the present invention, the additive according to the present invention, the cell according to the present invention, and the battery according to the present invention, and to the FIGURE and the description of the FIGURE.

### DETAILED DESCRIPTION OF THE INVENTION

Further advantages and advantageous embodiments of the subject matters according to the present invention are illustrated by the drawing and the exemplary embodiments, and explained in the following description. It is pointed out that the drawing and the exemplary embodiments are only descriptive in nature, and are not intended to limit the present invention in any way.

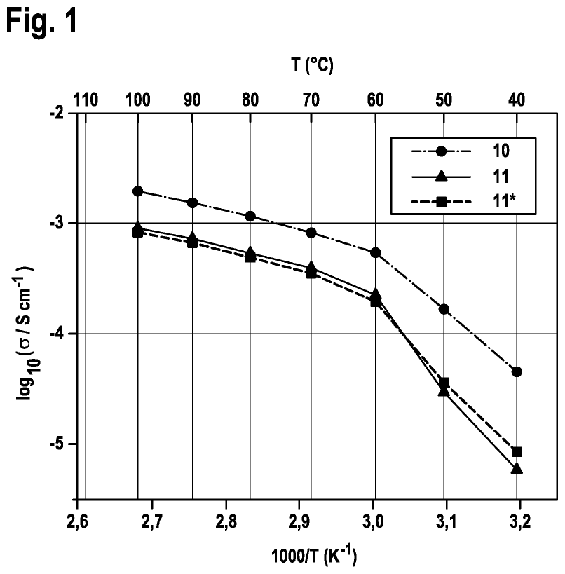

FIG. 1 shows a schematic graph for depicting the lithium ion conductivity of lithium [(trifluoromethyl)sulfonyl][(methylethylamide)sulfonyl]azanide in polyethylene oxide as a function of the temperature.

### EXEMPLARY EMBODIMENT 1: PREPARATION OF METHYLETHYLAMMONIUM [(TRIFLUOROMETHYL)SULFONYL][(METHYLETHYLAMIDE)SULFONYL]AZANIDE

Methylethylammonium [(trifluoromethyl)sulfonyl][(methylethylamide)sulfonyl]azanide was prepared by reacting [(trifluoromethyl)sulfonyl][chlorosulfonyl]imide, for example N-[(trifluoromethyl)sulfonyl]-N-[chlorosulfonyl]imide, with methylethylamine according to the following reaction scheme 1′:

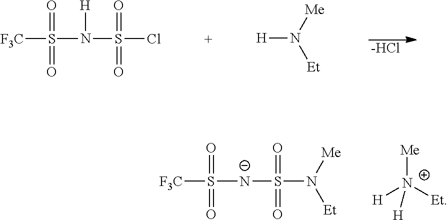

It was thus possible to prepare methylethylammonium [(trifluoromethyl)sulfonyl][(methylethylamide)sulfonyl]azanide in a particularly simple manner.

### EXEMPLARY EMBODIMENT 2: PREPARATION OF LITHIUM [(TRIFLUOROMETHYL)SULFONYL][(METHYLETHYLAMIDE)SULFONYL]AZANIDE WITH THE AID OF A ONE-POT REACTION

The reaction product of exemplary embodiment 1, methylethylammonium [(trifluoromethyl)sulfonyl][(methylethylamide)sulfonyl]azanide, was reacted with lithium hydroxide in a so-called one-pot reaction according to the following reaction scheme 2*′:

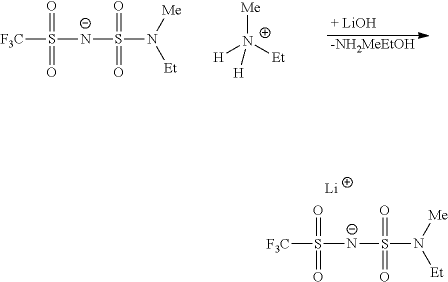

to give lithium [(trifluoromethyl)sulfonyl][(methylethylamide)sulfonyl]azanide. It was thus possible to prepare lithium [(trifluoromethyl)sulfonyl][(methylethylamide)sulfonyl]azanide in a particularly simple manner, for example in a purity or yield of greater than 90%.

### EXEMPLARY EMBODIMENT 3: PREPARATION OF POTASSIUM [(TRIFLUOROMETHYL)SULFONYL][(METHYLETHYLAMIDE)SULFONYL]AZANIDE WITH THE AID OF A ONE-POT REACTION

The reaction product from exemplary embodiment 1, methylethylammonium [(trifluoromethyl)sulfonyl][(methylethylamide)sulfonyl]azanide, was reacted with potassium hydroxide in a so-called one-pot reaction according to the following reaction scheme 2*″:

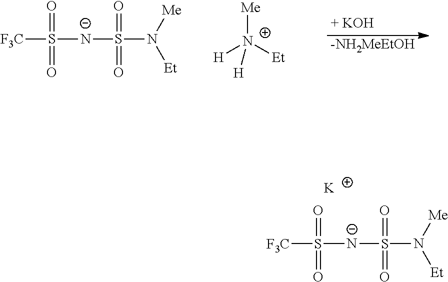

to give potassium [(trifluoromethyl)sulfonyl][(methylethylamide)sulfonyl]azanide. It was thus possible to prepare potassium [(trifluoromethyl)sulfonyl][(methylethylamide)sulfonyl]azanide in a particularly simple manner, for example in a purity or yield of greater than 88%.

### EXEMPLARY EMBODIMENT 4: PREPARATION OF HYDROGEN [(TRIFLUOROMETHYL)SULFONYL][(METHYLETHYLAMIDE)SULFONYL]AMINE

The reaction product from exemplary embodiment 1, methylethylammonium [(trifluoromethyl)sulfonyl][(methylethylamide)sulfonyl]azanide, was reacted with hydrochloric acid according to the following reaction scheme 2′:

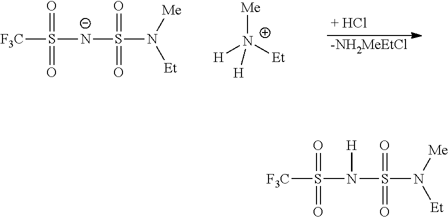

to give hydrogen [(trifluoromethyl)sulfonyl][(methylethylamide)sulfonyl]amine. It was thus possible to prepare hydrogen [(trifluoromethyl)sulfonyl][(methylethylamide)sulfonyl]amine in a particularly simple manner.

### EXEMPLARY EMBODIMENT 5: PREPARATION OF LITHIUM [(TRIFLUOROMETHYL)SULFONYL][(METHYLETHYLAMIDE)SULFONYL]AZANIDE WITH THE AID OF A MULTISTEP SYNTHESIS

The reaction product from exemplary embodiment 4, hydrogen [(trifluoromethyl)sulfonyl][(methylethylamide)sulfonyl]amine, was reacted with lithium carbonate according to the following reaction scheme 3′:

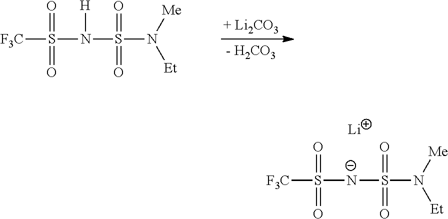 [SMILES: CCN(C)S(=O)(=O)NS(=O)(=O)C(F)(F)F ; "- H2CO3" ; [Li+] ; CCN(C)S(=O)(=O)[N-]S(=O)(=O)C(F)(F)F]

to give lithium [(trifluoromethyl)sulfonyl][(methylethylamide)sulfonyl]azanide. It was thus possible to prepare the lithium [(trifluoromethyl)sulfonyl][(methylethylamide)sulfonyl]azanide in a particularly high purity or yield, for example in a purity or yield of greater than 99%.

### EXEMPLARY EMBODIMENT 6: PREPARATION OF POTASSIUM [(TRIFLUOROMETHYL)SULFONYL][(METHYLETHYLAMIDE)SULFONYL]AZANIDE WITH THE AID OF A MULTISTEP SYNTHESIS

The reaction product from exemplary embodiment 4, hydrogen [(trifluoromethyl)sulfonyl][(methylethylamide)sulfonyl]amine, was reacted with potassium carbonate according to the following reaction scheme 3″:

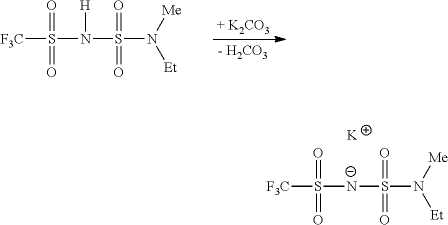 [SMILES: CCN(C)S(=O)(=O)NS(=O)(=O)C(F)(F)F ; "- H2CO3" ; [K+] ; CCN(C)S(=O)(=O)[N-]S(=O)(=O)C(F)(F)F]

to give potassium [(trifluoromethyl)sulfonyl][(methylethylamide)sulfonyl]azanide. It was thus possible to prepare the potassium [(trifluoromethyl)sulfonyl][(methylethylamide)sulfonyl]azanide in a particularly high purity or yield, for example in a purity or yield of greater than 99%.

### Properties of Lithium [(trifluoromethyl)sulfonyl][(methylethylamide)sulfonyl]azanide

The proton NMR of lithium [(trifluoromethyl)sulfonyl][(methylethylamide)sulfonyl]azanide showed a quartet at 3.04 ppm with an integral of two hydrogen atoms, a singlet at 2.65 ppm with an integral of 3 hydrogen atoms, and a triplet at 1.12 ppm with an integral of three hydrogen atoms.

The fluorine NMR of lithium [(trifluoromethyl)sulfonyl][(methylethylamide)sulfonyl]azanide showed a singlet at −79.03 ppm.

Lithium [(trifluoromethyl)sulfonyl][(methylethylamide)sulfonyl]azanide was stable in acidic and basic media at room temperature and at 80° C. over a period of 7 days.

Dissolved in propylene carbonate, lithium [(trifluoromethyl)sulfonyl][(methylethylamide)sulfonyl]azanide showed an anode stability of 4.6 V with respect to in particular metallic lithium (Li⁺/Li).

A 0.1 M solution of lithium [(trifluoromethyl)sulfonyl][(methylethylamide)sulfonyl]azanide in dimethyl ether (DME) had a conductivity of 4.3·10⁻⁵ S/cm at room temperature.

As the only salt in polyethylene oxide (PEO), with a concentration of 1 lithium ion per 20 ethylene oxide units, the conductivity of lithium [(trifluoromethyl)sulfonyl][(methylethylamide)sulfonyl]azanide was 5.38·10⁻⁴ S/cm. Under these conditions, lithium bis[(trifluoromethyl)sulfonyl]azanide (LiTFSI) showed a conductivity of 1.15·10⁻³ S/cm.

FIG. 1 shows a schematic graph for depicting the lithium ion conductivity of mixtures 11, 11* of lithium [(trifluoromethyl)sulfonyl][(methylethylamide)sulfonyl]azanide, prepared via two different synthesis routes, and polyethylene oxide with a concentration of 1 lithium ion per 20 ethylene oxide units as a function of the temperature, compared to a corresponding mixture of lithium bis[(trifluoromethyl)sulfonyl]azanide (LiTFSI) in polyethylene oxide 10 under the same conditions.

FIG. 1 shows that lithium [(trifluoromethyl)sulfonyl][(methylethylamide)sulfonyl]azanide has a high lithium ion conductivity and a similar temperature dependency of the lithium ion conductivity as for lithium bis[(trifluoromethyl)sulfonyl]azanide (LITFSI). Although the lithium ion conductivity of lithium [(trifluoromethyl)sulfonyl][(methylethylamide)sulfonyl]azanide is slightly lower than the lithium ion conductivity of lithium bis[(trifluoromethyl)sulfonyl]azanide (LITFSI), lithium [(trifluoromethyl)sulfonyl][(methylethylamide)sulfonyl]azanide advantageously has better temperature stability and high-voltage stability than lithium bis[(trifluoromethyl)sulfonyl]azanide (LITFSI), and also shows little or no corrosion of aluminum in contrast to lithium bis[(trifluoromethyl)sulfonyl]azanide (LITFSI).

What is claimed is:

1. A salt, comprising:
at least one alkali metal ion and/or at least one ammonium compound; and
at least one anion, the at least one anion containing:
at least one unfluorinated (N,N-dialkylamide)sulfonyl group and/or at least one unfluorinated (N,N-dialkylamide)sulfoximide group; and
at least one (perfluoroalkyl)sulfonyl group.

2. The salt as recited in claim 1, wherein the at least one anion has a general chemical formula of the following: 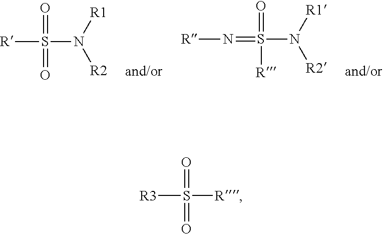
wherein:
R1 and R2 each independently stand for an unfluorinated alkyl group,
R′ stands for a substituent that includes at least one (perfluoroalkyl)sulfonyl group,
R1′ and R2′ each independently stand for an unfluorinated alkyl group,
(i) R″ stands for a substituent that includes at least one (perfluoroalkyl)sulfonyl group, and R′″ stands for a substituent that includes at least one unfluorinated, (N,N-dialkylamide)sulfoximide group and/or at least one, unfluorinated, (N,N-dialkylamide)sulfonyl group, or (ii) R″ stands for a substituent that includes at least one unfluorinated, (N,N-dialkylamide)sulfoximide group, and/or at least one unfluorinated, (N,N-dialkylamide)sulfonyl group, and R′″ stands for a substituent that includes at least one (perfluoroalkyl)sulfonyl group,
R3 stands for a perfluorinated alkyl group, and
R″″ stands for a substituent that includes at least one unfluorinated (N,N-dialkylamide)sulfonyl group and/or at least one unfluorinated (N,N-dialkylamide)sulfoximide group.

3. The salt as recited in claim 2, wherein R′ and/or R″ and/or R′″ and/or R″″ have at least one negative charge in the form of a bridging azanide group.

4. The salt as recited in claim 1, wherein the salt includes or is
a lithium salt that contains at least one lithium ion, and/or
a potassium salt that contains at least one potassium ion, and/or
a sodium salt that contains at least one sodium ion, and/or
an ammonium compound salt that contains at least one ammonium-compound, in particular a salt of an organic ammonium compound that contains at least one organic ammonium compound, and/or
an ammonium salt that contains at least one ammonium ion.

5. The salt as recited in claim 1, wherein the salt is a lithium salt that contains at least one lithium ion.

6. The salt as recited in claim 2, wherein:
R1 and R2 each independently stand for an unfluorinated methyl group or for an unfluorinated ethyl group or for an unfluorinated propyl group or for an unfluorinated butyl group, or for an unfluorinated methyl group or for an unfluorinated ethyl group, and
R1′ and R2′ each independently stand for an unfluorinated methyl group or for an unfluorinated ethyl group or for an unfluorinated propyl group or for an unfluorinated butyl group, or for an unfluorinated methyl group or for an unfluorinated ethyl group.

7. The salt as recited in claim 2, wherein:
(i) R1 and R2 stand for different unfluorinated alkyl groups, R1 standing for an unfluorinated methyl group and R2 standing for an unfluorinated ethyl group, or (ii) R1 and R2 stand for the same unfluorinated alkyl group, R1 and R2 each standing for an unfluorinated methyl group or R1 and R2 each stand for an unfluorinated ethyl group; and
(i) R1′ and R2′ stand for different unfluorinated alkyl groups, R1′ standing for an unfluorinated methyl group and R2′ standing for an unfluorinated ethyl group, or (ii) R1′ and R2′ stand for the same unfluorinated alkyl group, R1′ and R2′ each standing for an unfluorinated methyl group or R1′ and R2′ each stand for an unfluorinated ethyl group.

8. The salt as recited in claim 2, wherein R3 stands for a perfluorinated methyl group or for a perfluorinated ethyl group or for a perfluorinated propyl group or for a perfluorinated butyl group, or for a perfluorinated methyl group, or for a trifluoromethyl group.

9. The salt as recited in claim 1, wherein the at least one anion contains:
at least two unfluorinated (N,N-dialkylamide)sulfonyl groups, and/or
at least two unfluorinated (N,N-dialkylamide)sulfoximide groups; and/or
at least two (perfluoroalkyl)sulfonyl groups.

10. The salt as recited in claim 2, wherein the at least one anion has the general chemical formula:

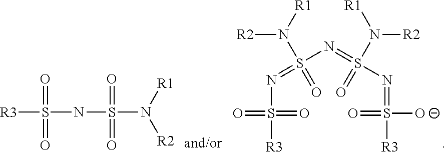

11. The salt as recited in claim 2, wherein the salt has the general chemical formula

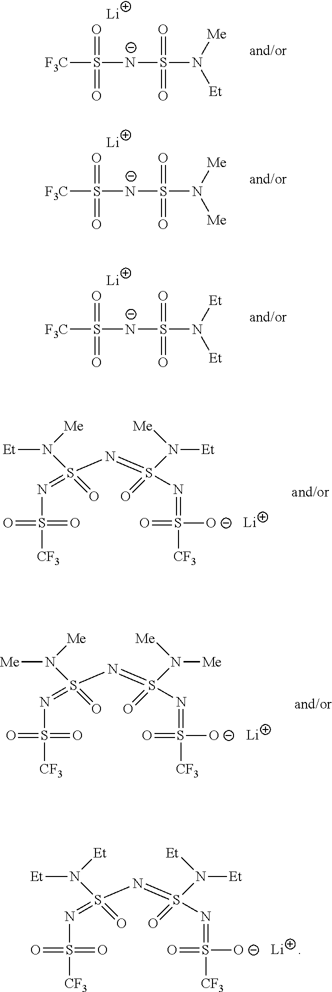

12. An electrolyte that includes at least one salt, the salt comprising:
at least one alkali metal ion and/or at least one ammonium compound; and
at least one anion, the at least one anion containing:
at least one unfluorinated (N,N-dialkylamide)sulfonyl group and/or at least one unfluorinated (N,N-dialkylamide)sulfoximide group; and
at least one (perfluoroalkyl)sulfonyl group.

13. The electrolyte as recited in claim 12, wherein the electrolyte includes at least one further salt, at at least one further salt including at least one further lithium salt or at least one lithium conducting salt.

14. The electrolyte as recited in claim 12, wherein the electrolyte is a polymer electrolyte.

15. An additive for an alkali metal cell, the alkali metal cell being a lithium cell, the additive including at least one salt, the at least one salt comprising:
at least one alkali metal ion and/or at least one ammonium compound; and
at least one anion, the at least one anion containing:
at least one unfluorinated (N,N-dialkylamide)sulfonyl group and/or at least one unfluorinated (N,N-dialkylamide)sulfoximide group; and
at least one (perfluoroalkyl)sulfonyl group.

16. An alkali metal cell, the alkali metal cell including lithium cell including an anode, a cathode, and a separator situated between the anode and the cathode, the separator including at least one salt comprising:
at least one alkali metal ion and/or at least one ammonium compound; and
at least one anion, the at least one anion containing:
at least one unfluorinated (N,N-dialkylamide)sulfonyl group and/or at least one unfluorinated (N,N-dialkylamide)sulfoximide group; and
at least one (perfluoroalkyl)sulfonyl group.

17. The alkali metal cell as recited in claim 16, wherein the cathode and/or the separator includes at least one polymer electrolyte including the salt.

18. The alkali metal cell as recited in claim 16, wherein the cathode includes at least one cathode active material having a cathode potential of ≥3.5 V, with respect to lithium, and/or the anode includes metallic lithium.

19. An alkali metal battery, at alkali metal battery being a lithium battery, the alkali metal battery including at least one cell including an anode, a cathode, and a separator situated between the anode and the cathode, the separator including at least one salt comprising:
at least one alkali metal ion and/or at least one ammonium compound; and
at least one anion, the at least one anion containing:
at least one unfluorinated (N,N-dialkylamide)sulfonyl group and/or at least one unfluorinated (N,N-dialkylamide)sulfoximide group; and
at least one (perfluoroalkyl)sulfonyl group.

20. A method for preparing a salt, comprising:
reacting a compound containing at least one (perfluoroalkyl)sulfonyl group and an electrophilic sulfur atom, with at least one unfluorinated N,N-dialkylamine, a reaction with at least one lithium salt, being subsequently carried out.
